(19) United States
(12) Patent Application Publication
(10) Pub. No.: US 2026/0041329 A1
(43) Pub. Date: Feb. 12, 2026

(54) SYSTEM AND METHOD OF DETECTING TISSUE PROXIMITY UTILIZING A TETRAPOLAR ELECTRODE ARRANGEMENT

(71) Applicant: St. Jude Medical, Cardiology Division, Inc., St. Paul, MN (US)

(72) Inventors: Hugo Andres Belalcazar, Minneapolis, MN (US); Isaac Remer, Richfield, MN (US); Derek C. Sutermeister, Ham Lake, MN (US); Gregory K. Olson, Elk River, MN (US)

(21) Appl. No.: 19/290,783

(22) Filed: Aug. 5, 2025

Related U.S. Application Data
(60) Provisional application No. 63/679,909, filed on Aug. 6, 2024.

(51) Int. Cl. A61B 5/0537; A61B 5/00; A61B 5/271

(52) U.S. Cl. CPC A61B 5/0537; A61B 5/271; A61B 5/742

(57) ABSTRACT

A medical device system includes a medical device, drive circuitry, and sense circuitry. The medical device includes a proximal end, a distal end, a pair of drive electrodes located at the distal end, and a first pair of sense electrodes located at the distal end, the first pair of sense electrodes being separate from the pair of drive electrodes. The drive circuitry is configured to provide a drive signal to the pair of drive electrodes. The sense circuitry is connected to the first pair of sense electrodes to sense a voltage generated in response to the drive signal provided to the pair of drive electrodes and to calculate a first tetrapolar measurement in response to the sensed voltage, the first tetrapolar measurement being indicative of tissue proximity, contact status, and/or orientation of the distal end of the medical device.

### CROSS-REFERENCE TO RELATED APPLICATION

This application claims benefit of priority to U.S. Provisional Patent Application No. 63/679,909 filed on Aug. 6, 2024, the entire disclosure of which is hereby incorporated by reference.

### TECHNICAL FIELD

The subject matter disclosed herein relates to medical devices and, in particular, to systems and methods of measuring an impedance associated with one or more electrodes.

### BACKGROUND

Impedance measured between a pair of electrodes located on a medical device may be utilized to detect contact between the electrodes and tissue. A drive signal is applied to the pair of electrodes and a voltage-sensing circuitry connected to one or both of the pair of electrodes is utilized to monitor the impedance between the pair of electrodes. The magnitude of the monitored impedance (or changes from a baseline value) can be utilized to detect whether the pair of electrodes is in contact with tissue.

In would be desirable to continue to improve on the specificity and sensitivity of systems and methods of detecting tissue proximity based on monitored electrical signals.

### SUMMARY

In some aspects, the techniques described herein relate to a medical device system including: a medical device including: a proximal end; a distal end; a pair of drive electrodes located at the distal end; and a first pair of sense electrodes located at the distal end, the first pair of sense electrodes being separate from the pair of drive electrodes; drive circuitry configured to provide a drive signal to the pair of drive electrodes; and sense circuitry connected to the first pair of sense electrodes to sense a voltage generated in response to the drive signal provided to the pair of drive electrodes and to calculate a first tetrapolar measurement in response to the sensed voltage, the first tetrapolar measurement being indicative of tissue proximity, contact status, and/or orientation of the distal end of the medical device.

In some aspects, the techniques described herein relate to a medical device system, wherein the first tetrapolar measurement is displayed to a user.

In some aspects, the techniques described herein relate to a medical device system, wherein the first tetrapolar measurement is displayed to the user as a signal trace that is color-coded to provide a visual indication of contact status.

In some aspects, the techniques described herein relate to a medical device system, further including: a tissue proximity detector configured to generate an output representative of tissue proximity, contact status, and/or orientation of the distal end based on the first tetrapolar measurement.

In some aspects, the techniques described herein relate to a medical device system, wherein the tissue proximity detector generates outputs representative of tissue proximity, contact status, and/or orientation of the distal end based on comparisons of the first tetrapolar measurement to one or more thresholds.

In some aspects, the techniques described herein relate to a medical device system, further including a second pair of sense electrodes utilized to generate a second tetrapolar measurement, wherein the tissue proximity detector generates an output representative of tissue proximity, contact status, and/or orientation of the distal end based on a comparison of the first tetrapolar measurement to the second tetrapolar measurement.

In some aspects, the techniques described herein relate to a medical device system, wherein the drive circuitry is a constant current source and the first tetrapolar measurement is based on the sensed voltage.

In some aspects, the techniques described herein relate to a medical device system, wherein the drive circuitry is a variable current source that delivers a variable current to the pair of drive electrodes, and wherein the first tetrapolar measurement is a tetrapolar impedance measurement calculated based on the sensed voltage and a magnitude of the variable current.

In some aspects, the techniques described herein relate to a medical device system, wherein the first pair of sense electrodes includes a first sense electrode and a second sense electrode, wherein the second sense electrode is located adjacent to the first sense electrode.

In some aspects, the techniques described herein relate to a medical device system, wherein the first pair of drive electrodes includes a first drive electrode and a second drive electrode, the first drive electrode located on a first side of the first pair of sense electrodes and the second drive electrode located on a second side of the first pair of sense electrodes.

In some aspects, the techniques described herein relate to a medical device system, wherein the first pair of drive electrodes includes a first drive electrode and a second drive electrode, wherein the second drive electrode is located adjacent to the first drive electrode.

In some aspects, the techniques described herein relate to a medical device system, wherein the second drive electrode is located adjacent to the first sense electrode.

In some aspects, the techniques described herein relate to a medical device system, wherein the medical device further includes one or more additional pairs of sense electrodes and wherein the sense circuitry is additionally connected to the one or more additional pairs of sense electrodes to measure additional tetrapolar measurements in response to the drive signal provided to the first pair of drive electrodes.

In some aspects, the techniques described herein relate to a medical device system, wherein the medical device includes a circular distal end, wherein the pair of drive electrodes and the pair of sense electrodes are located on the circular distal end.

In some aspects, the techniques described herein relate to a medical device system, wherein the medical device includes a basket distal end included of a plurality of splines, wherein the pair of drive electrodes and the pair of sense electrodes are located on one or more of the plurality of splines.

In some aspects, the techniques described herein relate to a medical device system, wherein the drive electrodes and the sense electrodes are arranged in a configuration having known distances between the drive electrodes and the sense electrodes.

In some aspects, the techniques described herein relate to a medical device system including: a medical device including: a first spline having at least a first pair of drive electrodes and a first pair of sense electrodes; and a second spline having at least a second pair of drive electrodes and a second pair of sense electrodes; and drive circuitry configured to provide a first drive signal to the first pair of drive electrodes and a second drive signal to the second pair of drive electrodes; and sense circuitry connected to the first pair of sense electrodes to measure a first tetrapolar measurement generated in response to the first drive signal provided to the first pair of drive electrodes, the sense circuitry connected to the second pair of sense electrodes to measure a second tetrapolar measurement generated in response to the second drive signal provided to the second pair of drive electrodes.

In some aspects, the techniques described herein relate to a medical device system, wherein the first and second tetrapolar measurements are displayed to a user.

In some aspects, the techniques described herein relate to a medical device system, wherein the first and second tetrapolar measurements are displayed to the user as first and second signal traces, respectively, wherein the first and second signal traces are color-coded to provide a visual indication of proximity, contact status, and/or orientation of the medical device.

In some aspects, the techniques described herein relate to a medical device system, further including: a tissue proximity detector configured to determine tissue proximity based on the first and second tetrapolar measurements.

In some aspects, the techniques described herein relate to a medical device, wherein the tissue proximity detector determines tissue proximity, contact status, and/or orientation of the medical device based, at least in part, on comparisons of the first tetrapolar measurement and the second tetrapolar measurement to one or more threshold values.

In some aspects, the techniques described herein relate to a medical device, wherein the tissue proximity detector determines tissue proximity, contact status, and/or orientation of the medical device based, in addition, on comparisons of the first tetrapolar measurement to the second tetrapolar measurement.

In some aspects, the techniques described herein relate to a medical device, wherein the first pair of drive electrodes and the first pair of sense electrodes are arranged in a first configuration and wherein the second pair of drive electrodes and the second pair of sense electrodes are arranged in a second configuration different from the first configuration.

In some aspects, the techniques described herein relate to a medical device, wherein the first pair of drive electrodes are located adjacent to one another and the first pair of sense electrodes are located adjacent to one another.

In some aspects, the techniques described herein relate to a medical device, wherein the first pair of sense electrodes are located adjacent to one another and the first pair of drive electrodes are separated from one another and located on opposite sides of the first pair of sense electrodes.

In some aspects, the techniques described herein relate to a method of detecting tissue proximity of a medical device, the method including: applying a drive signal to a first pair of drive electrodes located on the medical device; measuring a voltage between a first pair of sense electrodes located on the medical device, wherein the first pair of sense electrodes are separate from the first pair of drive electrodes, wherein the first pair of sense electrodes and the first pair of drive electrodes represent a tetrapolar configuration; calculating a first tetrapolar measurement based on the voltage measured between the first pair of sense electrodes; determining tissue proximity of the medical device based on the first tetrapolar measurement; and displaying the determined tissue proximity.

In some aspects, the techniques described herein relate to a method, wherein the tetrapolar measurement represents an impedance associated with a region adjacent to the tetrapolar configuration of drive electrodes and sense electrodes located on the medical device.

In some aspects, the techniques described herein relate to a method, further including: measuring a plurality of voltages associated with a plurality of pairs of sense electrodes; and calculating a plurality of tetrapolar measurements based on the plurality of measured voltages.

In some aspects, the techniques described herein relate to a method, wherein determining tissue proximity of the medical device further includes determining an orientation of the medical device based on the plurality of tetrapolar measurements.

In some aspects, the techniques described herein relate to a method, wherein determining tissue proximity of the first pair of sense electrodes includes calculating a distance from the tetrapolar configuration of drive electrodes and sense electrodes to the tissue based on a magnitude of the tetrapolar measurement.

In some aspects, the techniques described herein relate to a method, wherein displaying the determined tissue proximity to the user includes displaying the first tetrapolar measurement to a user as a first signal trace, wherein the first signal trace is color-coded to provide a visual indication of proximity to adjacent tissue.

### BRIEF DESCRIPTION OF THE DRAWINGS

FIG. 1 is a diagrammatic depiction of a system including a medical device for insertion within a patient, the system configured to utilize a two-terminal impedance measurement to determine proximity or contact status of the one or more electrodes located at a distal end of the medical device according to some embodiments.

FIG. 2 is a circuit diagram of a three-terminal impedance measurement circuit connected to first and second electrodes located on the medical device as known in the prior art.

FIGS. 3A and 3B are circuit diagrams illustrating tetrapolar measurement circuits according to some embodiments.

FIGS. 4A-4F are schematic views of a distal end of a medical device having an array of electrodes with various placements and geometries of the pair of drive electrodes and the pair of sense electrodes according to various embodiments.

FIG. 5A is a sensitivity map illustrating the sensitivity associated with an array of electrodes arranged in a tetrapolar alpha configuration according to some embodiments; FIG. 5B is a sensitivity map illustrating the sensitivity associated with an array of electrodes arranged in a tetrapolar beta configuration according to some embodiments.

FIG. 6A is a schematic illustrating the placement of a distal end of a medical device having an array of electrodes in proximity with tissue; FIG. 6B is a graph illustrating a three-terminal impedance measurement as compared with various configurations of tetrapolar measurements according to some embodiments.

FIG. 7A is a schematic illustrating the placement of a distal end of a medical device having an array of electrodes in proximity with tissue; FIG. 7B is a graph illustrating various configurations of tetrapolar measurements according to some embodiments.

FIG. 8 is a graph illustrating tetrapolar measurements collected while a distal end of a medical device is positioned within a patient according to some embodiments.

FIG. 9 is a schematic view of a distal end of a medical device having an array of electrodes including a first pair of drive electrodes and first and second pairs of sense electrodes according to some embodiments.

FIGS. 10 is a schematic view of a distal end of a medical device having a plurality of electrodes configured for tetrapolar measurements according to some embodiments.

FIG. 11 is a schematic view of a distal end of a medical device having a plurality of electrodes configured for tetrapolar measurements according to some embodiments.

FIG. 12 is a schematic view of a distal end of a medical device having a plurality of electrodes configured for tetrapolar measurements according to some embodiments.

FIG. 13 is a flowchart illustrating a method of utilizing tetrapolar measurements and utilizing the tetrapolar measurements to detect tissue proximity according to some embodiments.

FIGS. 14A and 14B are schematic views of a distal end of a medical device having a basket geometry that includes a plurality of splines and a single electrode on each spline configured for tetrapolar measurements according to some embodiments.

FIGS. 15A and 15B are schematic views of a distal end of a medical device having a plurality of splines, each spline including a plurality of electrodes configured for tetrapolar measurements according to some embodiments.

FIG. 16 is a flowchart illustrating a method of utilizing a combination of both tetrapolar measurements in combination with typical tissue impedance measurements (two-terminal or three-terminal measurements) to detect tissue proximity according to some embodiments.

### DETAILED DESCRIPTION

According to some embodiments, a tetrapolar electrode arrangement utilizes a pair of drive electrodes and a separate pair of sense electrodes. Drive circuitry is utilized to provide a drive current between the first pair of drive electrodes. Sense circuitry is utilized to measure the voltage or potential generated at the respective sense electrodes in response to the drive current, which in turn are utilized to generate a tetrapolar measurement. Voltages measured by the sense circuitry at the second pair of sense electrodes are utilized to detect tissue proximity of the catheter and/or of individual electrodes.

The sensitivity of the tetrapolar electrode arrangement is dependent, at least in part, on the placement and geometry of the respective current injection electrodes and the sense electrodes relative to one another. Conversely, the sensitivity of the tetrapolar electrode arrangement is not dependent on the surface area of the electrodes or on the resistance of the wires associated with the electrodes. Rather, the voltages measured at the pair of sense electrodes are a function of the materials (and associated conductivity) surrounding the tetrapolar electrode arrangement (e.g., tissue, blood pool, etc.). As a result, the tetrapolar measurement is not just indicative of whether the pair of sense electrodes are in contact with tissue, but indicative of the tissue proximity to the catheter in general (i.e., the presence of tissue within a region around the catheter and the plurality of electrodes making up the tetrapolar configuration).

In some embodiments, the tetrapolar measurement may be utilized for tissue proximity/contact. Because the sensitivity is dependent on the tetrapolar electrode arrangement, in some embodiments a plurality of sense electrodes is utilized to detect voltages generated in response to a single pair of drive electrodes, wherein the plurality of sense electrodes provide different levels of sensitivity based on the placement and geometry of the respective sense electrodes to the pair of drive electrodes.

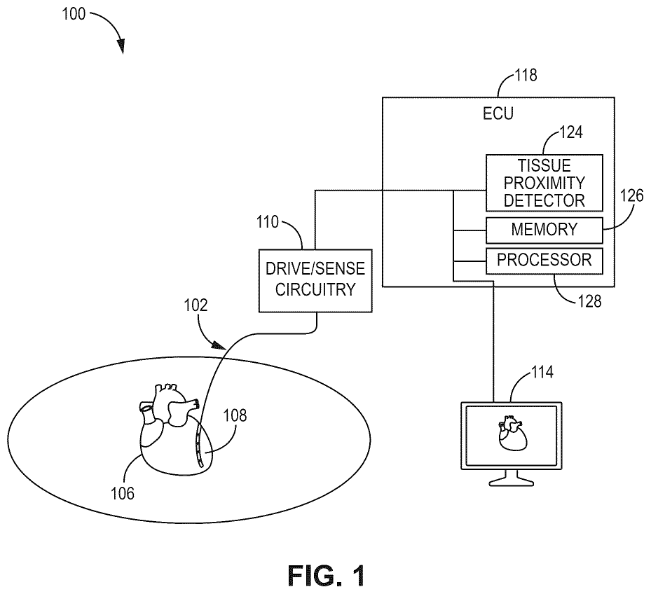

FIG. 1 is a diagrammatic depiction of a system 100 including a medical device 102 for insertion within a patient, the system configured to utilize a tetrapolar measurement to determine proximity or contact status between the one or more electrodes located at a distal end of the medical device and adjacent tissue according to some embodiments. In some embodiments, the system 100 includes a drive and sense circuitry 110, an electronic control unit (ECU) 112 includes memory 126 and a processor 128, wherein instructions stored by the memory 126 are executed by the processor 128 to implement tissue proximity detector 124. The system 100 may also include a display 114 for displaying information to the technician/doctor, which may include tissue proximity, contact status and/or orientation information derived from the tetrapolar measurements. In some embodiments, one or more reference electrodes (not shown) may be utilized. For example, in some embodiments the reference electrode is a surface patch electrode adhered to the skin of the patient.

In some embodiments, the medical device 102 is an elongate medical device, such as a diagnostic and/or therapy catheter, an introducer, sheath, or other similar type of device. The medical device 102 includes a distal end 108 and a proximal end (not shown) that includes a handle operated by a technician as well as interfaces for interfacing the medical device 102 to the drive/sense circuitry 110. The distal end 108 may include various sensors and/or components for localization/navigation of the distal end 108 within the patient, mapping of physiological parameters within the patient, and delivery of therapy. In particular, the distal end 108 of the medical device 102 includes a plurality of electrodes that may be utilized for one or more of these purposes.

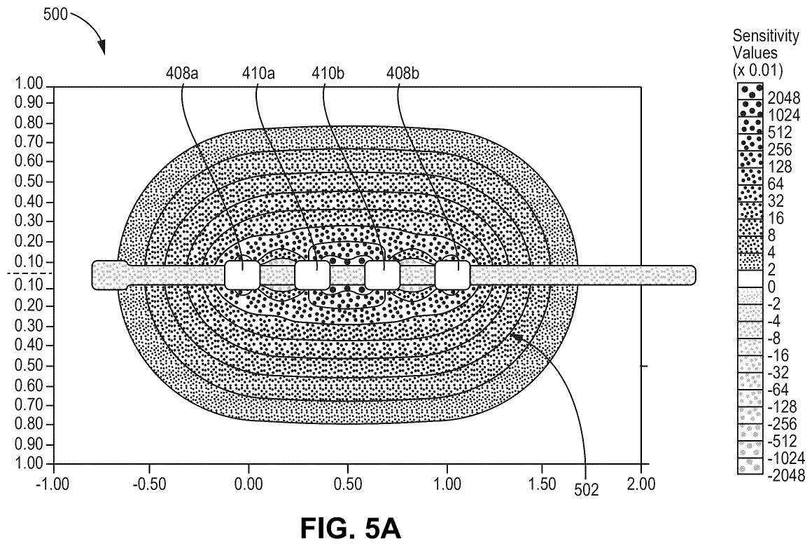

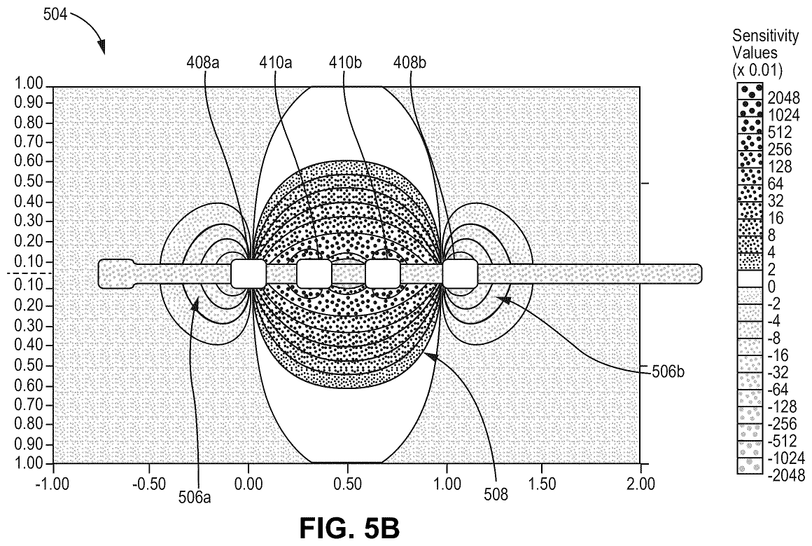

In some embodiments, tissue proximity and/or contact status of the distal end 104 of the medical device 102 is determined based on one or more electrical characteristics measured at the electrode. For example, in some embodiments the measured electrical characteristic is a tetrapolar impedance measurement generated in response to a drive or excitation signal being provided to a first pair of electrodes. The drive signal results in voltages being induced at one or more other electrodes (i.e., sense electrodes), which are sensed and utilized to determine tissue proximity and/or contact status. In some embodiments, tissue proximity detector 124 utilizes the tetrapolar measurement, either alone or in combination with other measured electrical characteristics, to determine the tissue proximity/contact status of the catheter. The term “tissue proximity” refers to information including distance of the catheter from adjacent tissue as well as orientation of the catheter to the adjacent tissue. As described in more detail below, the tetrapolar measurement responds to the introduction of tissue (e.g., resistive element) in the region surrounding the tetrapolar configuration of electrodes. The sensitivity of the tetrapolar measurement is dependent on the particular configuration of the sense electrodes and drive electrodes, as described in more detail below by the sensitivity maps shown in FIGS. 5A, 5B. In this way, tissue proximity is not limited to contact of any of the particular electrodes with adjacent tissue, but rather refers more broadly to the detection of tissue within a region surrounding the catheter and the tetrapolar configuration of electrodes. Tissue proximity may also refer to determining an orientation of the catheter relative to the adjacent tissue. In some embodiments, the term “contact status” is a binary determination, with the electrode either being “in contact” with the tissue or “not in contact” with the tissue. In other embodiments, the term “contact status” may include additional contact states, such as “intermittent contact”. In some embodiments, the tetrapolar measurements may be utilized for other applications, such as sheath detection (e.g., determination of whether the medical device and in particular the electrode located on the medical device is located within a sheath or external to the sheath).

In the embodiment shown in FIG. 1, drive/sense circuitry 110 is utilized to generate the drive signal. In some embodiments, drive/sense circuitry 110 generates one or more excitation or drive signals, each at a unique frequency. More specifically, the drive/sense circuitry 110 may generate a plurality of excitation or drive signals having unique frequencies within a range from about 100 Hz to over 500 kHz. In some embodiments, the drive signals are sinusoidal. In other embodiments, the drive signals may have other waveforms (e.g., square wave). In some embodiments, the unique frequencies are provided at a frequency less than 30 kHz, less than 20 kHz, and less than 10 kHz. In some embodiments, the unique frequencies are provided at a frequency less than 2 kHz. In some embodiments, the drive/sense circuitry includes a constant current source. In other embodiments, the drive/sense circuitry is a variable current source. The amplitude of the current provided by the drive signals is typically in the range of between 1-200 μA, and more typically about 5 μA, in one embodiment. In other embodiments, the drive/sense circuitry includes a voltage source for generating the desired drive signals. In still other embodiments, the drive/sense circuitry includes a constant voltage source for generating the desired drive signals. The drive/sense circuitry 110 may also generate signals involved in, for example, determining a location of the electrodes within the body of the patient that may be utilized for mapping, navigation, and/or therapy delivery. In some embodiments, a portion of the drive/sense circuitry 110 is implemented by ECU 112. In these embodiments, one or more digital-to-analog (D/A) converters (not shown) may be utilized to convert a digital signal generated by the ECU 112 to an analog signal delivered to the electrodes located at the distal end 108 of the medical device 102. In some embodiments, the drive signal may be applied to a plurality of different electrodes located at the distal end 108 and may require additional circuitry (not shown) such as a switch for selectively delivering the drive signal to the selected electrodes or modulator for generating drive signals at different frequencies.

In response to the drive signals supplied to the selected electrodes, the drive/sense circuitry 110 monitors a resulting voltage generated in response at selected sense electrodes. In some embodiments, a pair of sense electrodes are utilized, and the voltage is measured between the respective sense electrodes. In other embodiments, the voltage at each selected electrode is measured with respect to a reference electrode, such as a reference electrode associated with a surface patch or reference electrode located on the medical device 102 or separate medical device (not shown) located in the patient's body. In some embodiments, sensed voltages are converted to a digital signal by an analog-to-digital (A/D) converter (not shown) and provided to the ECU 112 for further processing. Once again, in some embodiments, additional components such as a switch and/or synchronous demodulator associated with the drive/sense circuitry 110 or ECU 112 may be utilized to select the sense electrodes or signals to be analyzed.

In some embodiments, the memory 126 may be configured to store data respective to the medical device 102, the patient, and/or other data (e.g., calibration data, configuration of drive electrodes/sense electrodes, locations relative to one another). Such data may be known before a medical procedure (medical device specific data, number of catheter electrodes, etc.), or may be determined and stored during a procedure. The memory 126 may also be configured to store instructions that, when executed by the processor 128, cause the ECU 118 to perform one or more methods, steps, functions, or algorithms described herein. For example, but without limitation, the memory 126 may include data and instructions for determining tetrapolar measurements respective of one or more electrodes on the medical device 102 and utilizing the tetrapolar measurements to determine proximity of the catheter to adjacent tissue, tissue contact between the catheter and adjacent tissue, orientation of the catheter relative to the adjacent tissue and/or other attributes of the medical device such as position within a sheath. In some embodiments, the tissue proximity detector 124 utilizes a processor executing instructions stored on the memory 126, an application specific integrated circuit (ASIC), or other type of processor. The ECU 112 may be connected to a display 114, which may display an output of sensed tissue (e.g., heart), the medical device (not shown) and/or determined contact status of the one or more electrodes of the medical device 102.

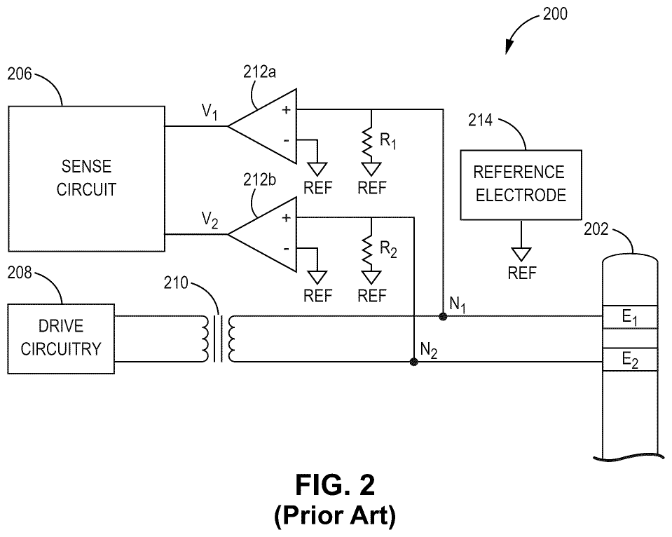

FIG. 2 is a circuit diagram of a three-terminal impedance measurement circuit 200 connected to first and second electrodes E₁, E₂ located on the medical device 202 as known in the prior art. The three-terminal impedance measurement circuit 200 includes sense circuitry 206, drive circuitry 208, isolation/transformer 210, first and second operational-amplifier (hereinafter amplifiers) 212a, 212b, and a reference electrode 214 (e.g., surface patch electrode). The drive circuitry 208 provides a drive current to electrodes E₁, E₂ and sense circuitry 206 measures the voltage V₁, V₂ at each electrode E₁, E₂, respectively. The measured voltages can be utilized to determine an impedance between the respective electrodes E₁, E₂ that in turn is utilized to determine, for example, contact status between the electrodes E1, E2 and adjacent tissue and/or sheath detection.

Drive circuitry 208 is connected to isolation/transformer 210 to generate a drive current. Sense circuitry 206 is connected to monitor the voltage between nodes N₁, N₂ and a reference node. In some embodiments, the reference node may be provided by a reference node located on the medical device 202 or a reference electrode affixed to the skin of the patient (i.e., surface patch electrode). First amplifier 212a has a non-inverting input connected to node N₁, which is associated with first electrode E₁, and has an inverting input connected to the reference node (denoted V_{ref}). The output V₁ provided at the output of the first amplifier 212a is reflective of the voltage between the first node N₁ and the reference node. The circuitry utilized to monitor the voltage between the first node N₁ and the reference node is referred to as a first channel. Second amplifier 212b has a non-inverting input connected to node N₂, which is associated with the second electrode E₂, and has an inverting input connected to the reference node (denoted V_{ref}). The output V₂ provided at the output of the second amplifier 212b is reflective of the voltage between the second node N₂ and the reference node. The circuitry utilized to monitor the voltage between the second node N₂ and the reference node is referred to as a second channel. By injecting current between electrodes E1 and E2 and measuring a voltage between the first node N₁ and a reference node and/or between the second node N₂ and a reference node an impedance can be calculated with respect to either the first electrode E₁ and/or the second electrode E₂. The impedance measurement may be referred to as a three-terminal impedance measurement, based on the current injected between two electrodes and the reference electrode utilized to in combination with one of the electrodes to measure a voltage.

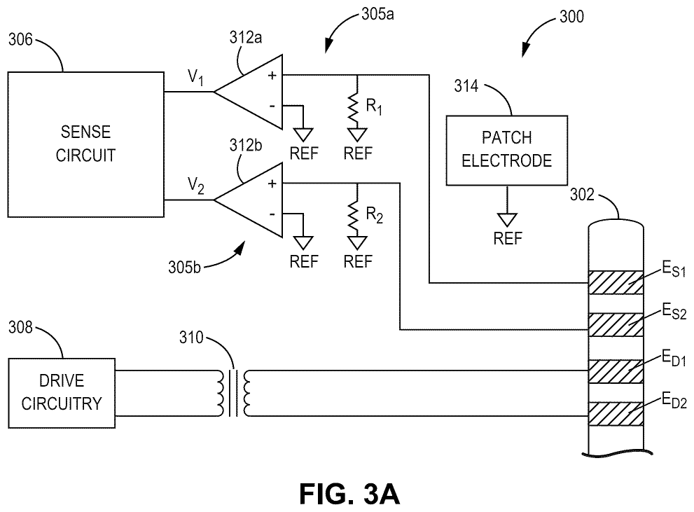

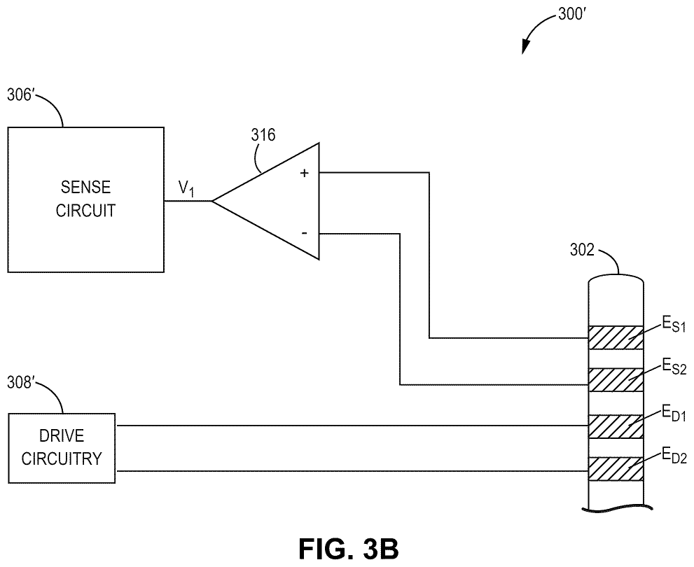

FIGS. 3A and 3B are circuit diagrams illustrating the tetrapolar measurement circuits 300 and 300′ according to some embodiments of the present invention. In the embodiment shown in FIG. 3A, the tetrapolar impedance measurement circuit 300 includes sense circuitry 306, drive circuitry 308, isolation/transformer 310, first and second operational amplifier (hereinafter amplifiers) 312a, 312b, and a reference electrode 314 (e.g., surface patch electrode). In this example, medical device 302 includes at least four electrodes E_{S1}, E_{S2}, E_{D1}, and E_{D2} located at a distal end of the device 302. The drive circuitry 308 provides a drive current to a first pair of electrodes referred to as the drive electrodes E_{D1}, E_{D2} and sense circuitry 306 measures the voltage between the sense electrodes E_{S1}, E_{S2}. As described above, in some embodiments the drive circuitry 308 is a constant current source capable of providing a constant current between the first and second drive electrodes E_{D1} and E_{D2}. In other embodiments the drive circuitry 308 is a variable current source. In some embodiments, a current sensor may be provided to sense the current provided to the first and second drive electrodes E_{D1} and E_{D2}, wherein the magnitude of the drive current is utilized in combination with voltage sensed between the first and second sense electrodes E_{S1}, E_{S2} to calculate the impedance in the region surrounding the configuration of electrodes, which will vary based on the presence of tissue within this region. The drive current provided to drive electrodes E_{D1} and E_{D2} creates a potential field that induces voltages at the sense electrodes E_{S1}, E_{S2} despite the sense electrodes not being directly connected to the drive circuitry 308. As described in more detail below, the position and geometry of the sense electrodes E_{S1} and E_{S2} relative to the drive electrodes E_{D1} and E_{D2} determine the sensitivity of the tetrapolar measurement circuit, while the resultant measurement is a function of the attributes (e.g., conductivity) of the surrounding environment (e.g., blood pool, tissue, sheath, etc.). For example, FIGS. 5A and 5B illustrate sensitivity profiles of two different configurations of drive electrodes and sense electrodes relative to one another. The use of sense electrodes E_{S1} and E_{S2} separate from the drive electrodes E_{D1} and E_{D2} makes the resultant measurements independent of the wires connecting the electrodes and/or the interface between the electrodes and the material around them.

In the embodiment shown in FIG. 3A, a first channel 305a includes an amplifier 312a having a non-inverting input connected to the first sense electrode Esi and an inverting input connected to a reference node associated with a reference electrode 314. Likewise, a second channel 305b includes an amplifier 312b having a non-inverting input connected to the second sense electrode E_{S2} and an inverting input connected to the reference node associated with a reference electrode 314. The amplifiers 312a, 312b generate an output V₁, V₂, respectively, representative of the voltage between the first and second sense electrodes E_{S1} and E_{S2}. For example, amplifier 312a generates an output V₁ representative of the voltage between the first sense electrode E_{S1} and the reference node and amplifier 312b generates an output V₂ representative of the voltage between the second sense electrode E_{S2} and the reference node. The outputs V₁ and V₂, because they are both measured with respect to the reference node, can be used to calculate the voltage between the first and second sense electrodes E_{S1} and E_{S2}. In some embodiments, based on the current supplied between the first and second drive electrodes E_{D1} and E_{D2}, the voltage measured between the first and second sense electrodes E_{S1} and E_{S2} can be utilized to calculate an impedance associated with the first and second sense electrodes E_{S1} and E_{S2}. This measurement is referred to herein as a tetrapolar measurement (sometimes referred to as a tetrapolar impedance measurement). The tetrapolar measurement may be analyzed by the sense circuitry 306 or may be communicated to the tissue proximity detector 124 implemented by the ECU 112 to make determinations regarding tissue proximity/contact. It should be noted, in some embodiments the drive current provided to the first and second drive electrodes E_{D1} and E_{D2} must be measured to calculate the impedance measurement associated with the first and second sense electrodes E_{S1} and E_{S2}. In other embodiments, a constant current source is utilized, and no measurement of the current is required.

In the embodiment shown in FIG. 3B, rather than measure the voltages between the first and second sense electrodes E_{S1}, E_{S2} and a reference node, the voltage between the first and second sense electrodes E_{S1}, E_{S2} is measured using voltage differential amplifier 316 having a first input connected to first sense electrode E_{S1}, a second input connected to the second sense electrode E_{S2}. In the embodiments shown in both FIGS. 3A and 3B, sense circuitry 306 and 306′ measure the difference in potential between the first sense electrode E_{S1} and the second sense electrode E_{S2}. In addition, the embodiment shown in FIG. 3B does not isolate the drive circuitry 308 from the drive electrodes with isolation transformer 310. Rather, in the embodiment shown in FIG. 3B the drive circuitry 308′ is connected directly to the first and second drive electrodes E_{D1} and E_{D2}.

Although the embodiments shown in FIGS. 3A and 3B illustrate a single tetrapolar measurement circuit (i.e., one pair of drive electrodes, one pair of sense electrodes), in other embodiments a plurality of tetrapolar measurement circuits may be employed on a single device. In these embodiments, a first drive signal is provided to a first pair of drive electrodes and voltages induced between a first pair of sense electrodes in response to the first drive signal are measured to form a first tetrapolar measurement. A second drive signal (operating at a different frequency than the first drive signal or time multiplexed with the first drive signal) is provided to a second pair of drive electrodes and voltages induced between a second pair of sense electrodes in response to the second drive signal are measured to form a second tetrapolar measurement.

In some embodiments, the tetrapolar measurements are communicated to the ECU 118 (as shown in FIG. 1) for analysis. In some embodiments, tissue proximity detector 124 analyzes the one or more tetrapolar measurements to detect tissue proximity, contact status, and/or orientation of the distal end of the catheter that includes the tetrapolar drive and sense electrodes relative to adjacent tissue. In some embodiments, the tissue proximity detector 124 compares the received tetrapolar measurements with one or more thresholds. As discussed in more detail below, different configurations of tetrapolar electrodes (i.e., different positions of drive electrodes and sense electrodes) provide different sensitivities, so tissue proximity detector 124 may also store information regarding the configurations of the tetrapolar electrodes and the thresholds to apply to each. In some embodiments, tissue proximity detector may also utilize combinations of tetrapolar measurements. For example, tissue proximity detector may compare a plurality of tetrapolar measurements to a plurality of threshold values (e.g., with threshold values selected based on the tetrapolar electrode configuration). In other embodiments, tissue proximity detector may compare two or more of the tetrapolar measurements to one another. Based on one or more of these comparisons, the tissue proximity detector makes determinations regarding tissue proximity, orientation, and/or contact of the catheter—in particular, the portion of the catheter including the tetrapolar electrode configuration—to the adjacent tissue.

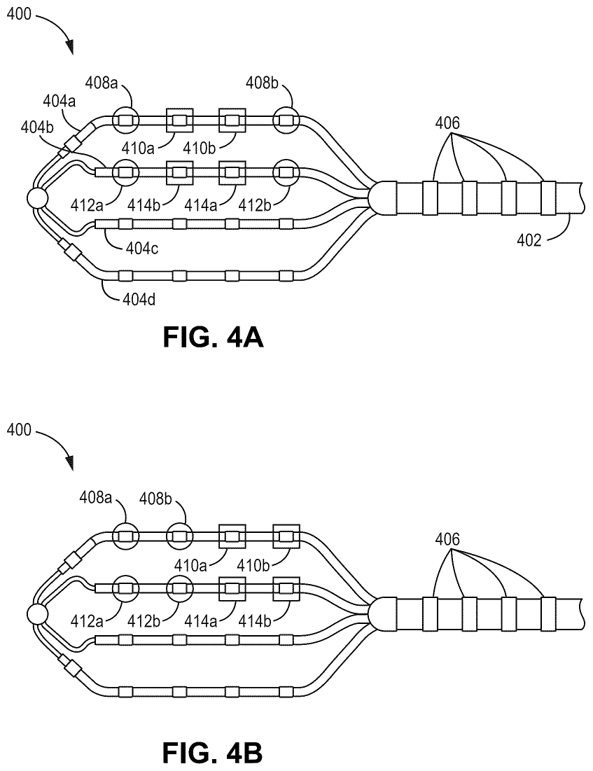

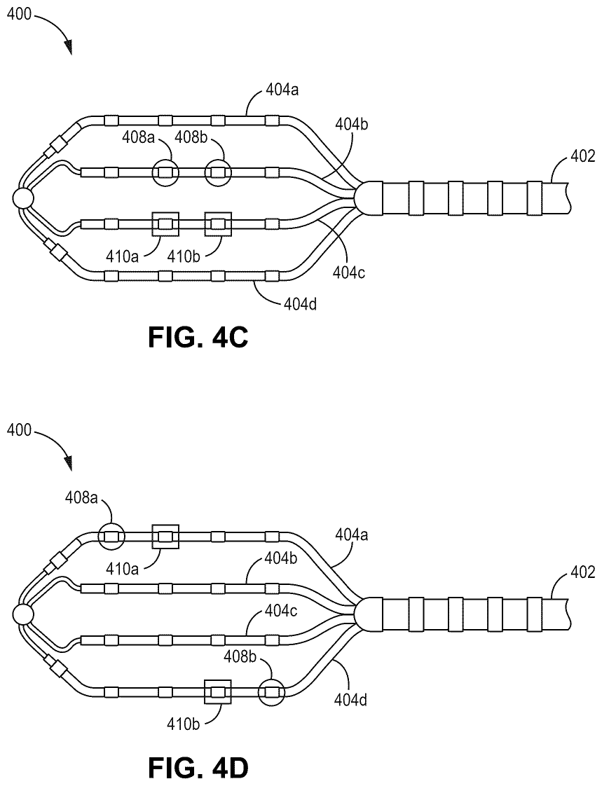

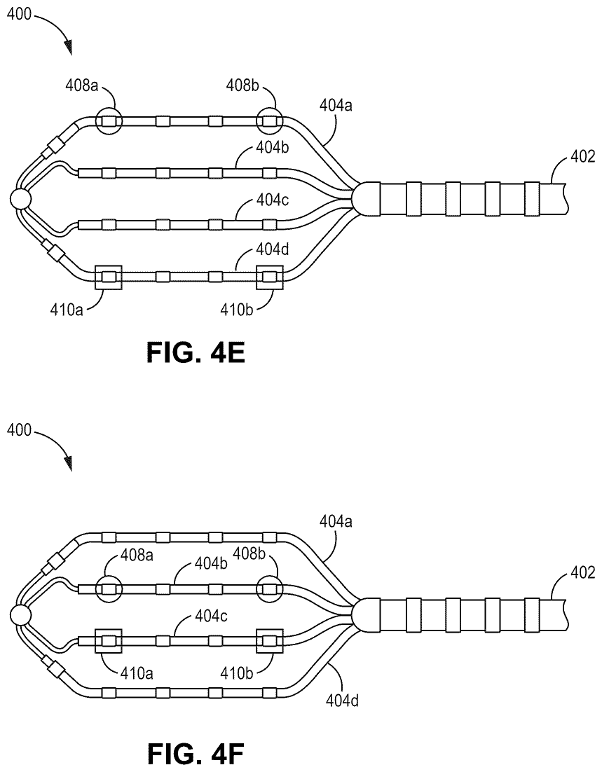

FIGS. 4A-4F are schematic views of a distal end 400 of a medical device having a planar array of electrodes with various placements and geometries of the pair of drive electrodes and the pair of sense electrodes according to various embodiments. In each of these examples, the distal end 400 of the medical device includes a shaft 402 having a plurality of shaft electrodes 406. The distal end 400 of the medical device is positioned at the distal end of the shaft 402 and in the embodiment shown in FIGS. 4A-4F the distal end is comprised of four splines 404a, 404b, 404c, and 404d. Each spline includes four electrodes positioned in a 4×4 array on the splines. In other embodiments, the number of splines and/or the number of electrodes on each spline may be varied along with the geometry of the distal end of the device. However, the geometry of the planar array of electrodes is well suited for tetrapolar measurements, in which changes to the location and geometry of the electrodes acting as the drive electrodes and the electrodes acting as the sense electrodes varies the sensitivity and/or specificity of the results. FIGS. 4A-4F illustrate non-exclusive examples of various locations and geometries of the sense electrodes and drive electrodes. In each of the embodiments shown in FIGS. 4A-4F, the sense electrodes are identified with squares drawn around the electrodes and the drive electrodes are identified by circles drawn around the electrodes. In each of these embodiments, sense electrodes may be interchanged with one another and the drive electrodes may be interchanged with one another.

For example, FIG. 4A illustrates a configuration in which the first and second sense electrodes 410a, 410b are located adjacent to one another on a first spline (e.g., spline 404a). The first drive electrode 408a is located on a first side of the first and second sense electrodes, adjacent to first sense electrode 410a and the second drive electrode 408b is located on a second side of the first and second sense electrodes, adjacent to second sense electrode 410b. For the purposes of description, this configuration of drive electrodes and sense electrodes is referred to herein as the tetrapolar alpha configuration. In this embodiment, both the first and second sense electrodes 410a, 410b and the first and second drive electrodes 408a, 408b are located on the same spline. In the embodiment shown in FIG. 4A the electrodes are located on spline 404a, but in other embodiments the electrodes may be located on any of the other spines 404b, 404c, and 404d. In some embodiments, maintaining the positions of the drive electrodes 408a, 408b and sense electrodes 410a, 410b relative to one another provides improved performance (i.e., arranging the drive electrodes and the sense electrodes in a configuration in which distances between each are known and relatively constant despite movement/bending of the electrode array).

Depending on the type of catheter or application on which the tetrapolar measurement circuit is utilized, it may be beneficial to select combinations of drive electrodes and sense electrodes in which positions will remain relatively unchanged relative to one another. For example, in the embodiment shown in FIG. 4A, it may be beneficial to locate the drive electrodes 408a, 408b and sense electrodes 410a, 410 on the same spline (e.g., spline 404a) as the relative distances between plurality of electrodes remains approximately unchanged despite bending/flexing of the distal tip.

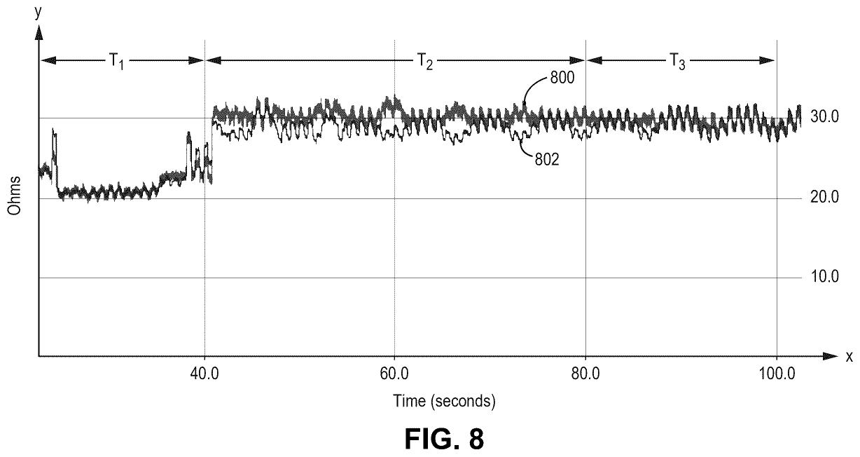

In some embodiments, more than one tetrapolar measurement circuit may be employed on a single device. For example, in the embodiment shown in FIG. 4A, a second tetrapolar measurement circuit utilizes drive electrodes 412a, 412b and sense electrodes 414a, 414b located on a second spline 404b. A second drive signal is provided to drive electrodes 412a, 412b. In some embodiments, the second drive signal is provided at a second frequency different than the frequency of the first drive signal so that the first drive signal and the second drive signal may be provided simultaneously. In other embodiments, the first drive signal provided to the first drive electrodes 408a, 408b may be time multiplexed with the second drive signal provided to the second drive electrodes 412a, 412b. In this embodiment, the first and second drive signals may be provided at the same frequency so long as the signals do not overlap in time with one another. As described in more detail with respect to FIG. 8, monitoring first and second tetrapolar measurements associated with first and second tetrapolar circuits (for example, as shown in FIG. 4A) may be utilized as an additional input to determine tissue proximity. For example, in some embodiments the first and second tetrapolar measurements may be compared to one or more thresholds to determine tissue proximity and in addition may be compared to one another to provide additional information regarding proximity to tissue.

Although not shown in FIG. 4A, in some embodiments, shaft electrodes 406 located on the shaft 402 may also be utilized to generate tetrapolar measurements. In the embodiment shown in FIG. 4A, four shaft electrodes 406 are illustrated, two of which would be utilized as drive electrodes and two of which would be utilized as sense electrodes. In some embodiments, the alpha configuration may be utilized with respect to the shaft electrodes 406 or some other configuration (e.g., beta configuration shown in FIG. 4B).

FIG. 4B illustrates a configuration in which the first and second sense electrodes 410a, 410b are located adjacent to one another on a first spline (e.g., spline 404a) and the first and second drive electrodes 408a, 408b are also located adjacent to one another on the first spline (e.g., spline 404a), with the second drive electrode 408b being located adjacent to the first sense electrode 410a. Once again, both the first and second sense electrodes 410a, 410b and the first and second drive electrodes 408a, 408b are located on the same spline (e.g., spline 404a). This configuration of drive electrodes and sense electrodes is referred to herein as the tetrapolar beta configuration. As described with respect to FIG. 4A, in some embodiments, additional tetrapolar measurement circuits may be utilized to generate a plurality of tetrapolar measurements with respect to the distal end 400 of the device. For example, a second tetrapolar beta configuration consisting of a second pair of sense electrodes and a second pair of drive electrodes may be employed on one of the other plurality of splines 404b, 404c, or 404d) to generate a second tetrapolar measurement.

FIG. 4C illustrates a configuration in which the first and second drive electrodes 408a, 408b are located adjacent to one another on a first spline (e.g., spline 404b) and first and second sense electrodes 410a, 410b are located adjacent to one another on a second spline (e.g., spline 404c). In this embodiment, the spline hosting the first and second sense electrodes 410a, 410b is located adjacent to the spline hosting the first and second drive electrodes 408a, 408b. This configuration of drive electrodes and sense electrodes is referred to herein as the tetrapolar small square configuration.

FIG. 4D illustrates a configuration in which the first and second drive electrodes 408a, 408b are located diagonally from one another. For example, first drive electrode 408a is located at the most distal location of the first spline 404a and the second drive electrode 408b is located at the most proximal location of the fourth spline 404d, positioned diagonally with respect to the first drive electrode 408a. The first sense electrode 410a is positioned adjacent to the first drive electrode 408a on the first spline 404a. The second sense electrode 410b is positioned adjacent to the second drive electrode 408b on the fourth spline 404d. This configuration of drive electrodes and sense electrodes is referred to as the tetrapolar narrow X configuration.

FIG. 4E illustrates a configuration in which the first and second drive electrodes 408a, 408b are located on opposite ends of a first spline. For example, first drive electrode 408a is located at the most distal location of the first spline 404a and the second drive electrode 408b is located at the most proximal location of the first spline 404a. In this configuration, one or more additional electrodes are located between the first and second drive electrodes. Likewise, the first and second sense electrodes 410a, 410b are located on opposite ends of a second spline. For example, the first sense electrode 410a is located at the most distal location of the fourth spline 404d and the second sense electrode 410b is located at the most proximal location of the fourth spline 404d. In this configuration, one or more additional electrodes are located between the first and second sense electrodes 410a, 410b. In this embodiment, the spline 404a hosting the first and second drive electrodes 408a, 408b is separated from the spline 404d hosting the first and second sense electrodes 410a, 410b by one or more intervening splines. This configuration of drive electrodes and sense electrodes is referred to as the tetrapolar square configuration.

FIG. 4F illustrates a configuration in which the first and second drive electrodes 408a, 408b are located on opposite ends of a first spline. For example, first drive electrode 408a is located at the most distal location of the second spline 404b and the second drive electrode 408b is located at the most proximal location of the second spline 404b. In this configuration, one or more additional electrodes are located between the first and second drive electrodes. Likewise, the first and second sense electrodes 410a, 410b are located on opposite ends of a second spline. For example, the first sense electrode 410a is located at the most distal location of the third spline 404c and the second sense electrode 410b is located at the most proximal location of the third spline 404c. In this configuration, one or more additional electrodes are located between the first and second sense electrodes 410a, 410b. In this embodiment, the spline 404a hosting the first and second drive electrodes 408a, 408b is located adjacent to the spline 404c hosting the first and second sense electrodes 410a, 410b. This configuration of drive electrodes and sense electrodes is referred to as the tetrapolar rectangular configuration.

In other embodiments, any of the tetrapolar configurations illustrated in FIGS. 4A-4F may be utilized alone or in combination with one another. For example, in some embodiments, a device may include a first tetrapolar configuration of drive and sense electrodes and a second tetrapolar configuration of drive and sense electrodes different from the first configuration.

Each of the configurations illustrated in FIGS. 4A-4F defines the sensitivity of the tetrapolar measurement. FIGS. 5A, 5B illustrate sensitivity maps associated with the tetrapolar alpha configuration (shown in FIG. 4A) and the tetrapolar beta configuration (shown in FIG. 4B).

FIG. 5A illustrates the sensitivity map 500 associated with the tetrapolar alpha configuration, which includes drive electrodes 408a, 408b and sense electrodes 410a, 410b. The sensitivity map includes positive regions and negative regions. The tetrapolar measurement increases in response to a resistive element (e.g., tissue) entering the positive regions and decreases in response to a resistive element entering the negative regions. The sensitivity map 500 illustrates a largely positive sensitivity profile 502 located around both the drive electrodes 408a, 408b and sense electrodes 410a, 410b. That is, the presence of a resistive element such as tissue within any of the positive sensitivity regions will result in an increase in the measured tetrapolar measurement. Compared with the sensitivity profile shown in FIG. 5B, the sensitivity profile shown in FIG. 5A is more homogenous (mostly positive sensitivity profile).

FIG. 5B illustrates the sensitivity map 504 associated with the tetrapolar beta configuration, which includes drive electrodes 408a, 408b and sense electrodes 410a, 410b, albeit in a different configuration. In contrast with the sensitivity map illustrated in FIG. 5A, the sensitivity map 504 includes negative regions 506a, 506b extending from opposite ends of the beta tetrapolar configuration (specifically, to the left of drive electrode 408a and to the right of sense electrode 410b). A resistive element moving into the negative sensitivity regions 506a or 506b will cause a decrease in the tetrapolar measurement. A resistive element moving into the positive sensitivity region 508 will cause an increase in the tetrapolar measurement. One advantage of the beta tetrapolar configuration of FIG. 5B is that a semi-planar tissue surface approaching the catheter at an angle will substantially intrude the negative sensitivity area, therefore yielding a negative or low response. This is desired, because such an angled position is not a full contact position of the catheter. Only when the catheter is fully flat against the tissue surface, will there be a net positive signal (since there is more positive sensitivity than negative in that position). In other words, the beta tetrapolar configuration is less sensitive than the alpha tetrapolar configuration to non-planar tissue approaches, a desirable characteristic to avoid false indications of contact in some embodiments.

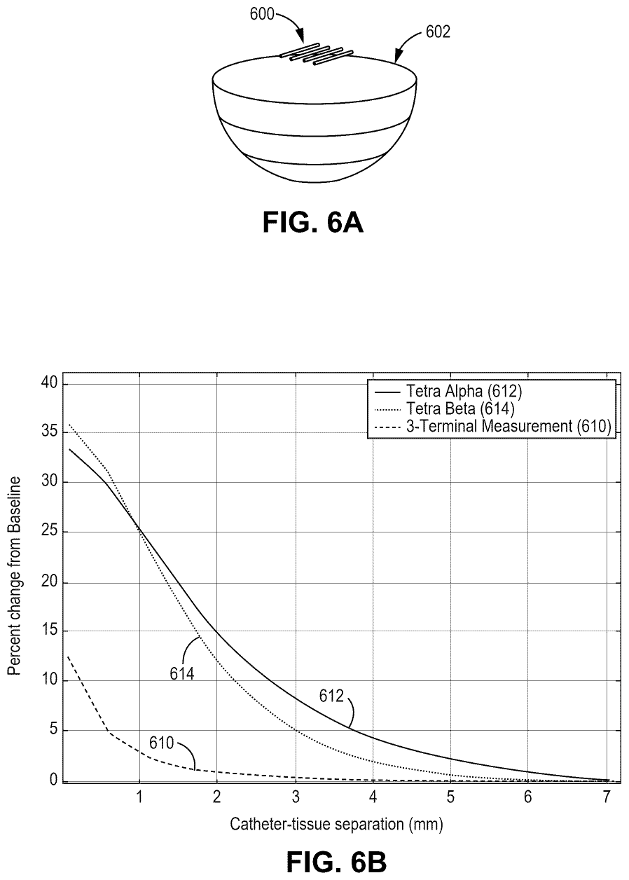

FIG. 6A is a schematic illustrating a simulated planar array of electrodes (such as those described with respect to FIGS. 4A-4F) placed at various positions relative to simulated tissue 602. Corresponding tetrapolar measurements utilizing the alpha configuration and the tetrapolar beta configuration are compared with traditional three-terminal measurements (generated, for example, using the circuit shown in FIG. 2 as known in the prior art) in the graph shown in FIG. 6B.

In the simulation, the simulated planar array 600 was arranged approximately parallel with the tissue surface 602 and the distance between the planar array 600 and the tissue surface 602 was modified. Traditional three-terminal impedance measurements taken at the various distances are illustrated by line 610, measurements taken using the tetrapolar alpha configuration are illustrated by line 612, and measurements taken using the tetrapolar beta configuration are illustrated by line 614.

The graph illustrated in FIG. 6B the y-axis illustrates the precent change in measured value from a baseline value and the x-axis illustrates the distance between the planar array 600 and the tissue 602. The three-terminal impedance measurement 610 illustrates that the percent change from the baseline increases as the distance between the planar array 600 and the tissue 602 decreases. However, the decrease is not linear. Rather the three-terminal impedance measurement increases much more rapidly as the electrode located on the planar array 600 approaches the tissue and decreases rapidly as the planar array 600 moves away from the tissue.

In contrast, both the tetrapolar alpha configuration illustrated by line 612 and the tetrapolar beta configuration illustrated by line 614 show improved performance. Both illustrate greater percent changes from baseline when the planar array 600 is in contact with the tissue 602 (e.g., tetrapolar alpha and beta measurements are equal to approximately 35% at tissue contact, as compared with the three-terminal measurement equal to approximately 13%). In addition, the decrease in the percent change from baseline as the planar array 600 moves away from the tissue 602 is more linear than the three-terminal impedance measurement 610. For example, the three-terminal impedance measurement shows a 5% change from the baseline at approximately 0.6 mm tissue separate whereas the tetrapolar beta configuration 614 shows a 5% change from the baseline at approximately 3 mm separation from the tissue 602 and the tetrapolar alpha configuration 612 shows a 5% change from the baseline at approximately 3.7 mm. In this way, the tetrapolar configurations demonstrate increased sensitivity (i.e., correctly identifying true positive rate or in the case of tissue contact, correctly identifying when the electrodes IS in contact with tissue) but a decrease in specificity as both the tetrapolar alpha and beta configurations generate responses to nearby tissue. In addition, the more linear response of the tetrapolar measurements as the electrode array is moved further away from the tissue allows a distance between the electrode array and the adjacent tissue to be calculated. In some embodiments, the tetrapolar measurements allow for more accurate determinations of distance between the electrode array and tissue, rather than merely a determination of whether the array is in contact with the tissue or not. In the embodiment shown in FIG. 6B, the tissue proximity detector 124 (shown in FIG. 1) may compare the tetrapolar measurements to a plurality of threshold values to determine a distance between the electrode array and the adjacent tissue. For example, if the percent change in the tetrapolar measurement from a baseline value is equal to or greater than 33%, then a determination is made that the electrode array is in contact with the tissue. If the percent change is equal to 25%, a determination is made that the electrode array is 1 mm from the adjacent tissue. If the percent change is equal to 15%, a determination is made that the electrode array is 2 mm from the adjacent tissue, and so on. Various configurations of tetrapolar circuits may be capable of detecting tissue proximity at various distances, greater than the embodiments shown in FIGS. 6A, 6B. In this way, the tissue proximity detector 124 may generate as an output a tissue contact status indicating whether the portion of the catheter associated with the tetrapolar electrode configuration (alpha or beta configuration) is in contact with the adjacent tissue. In addition, in some embodiments the tissue proximity detector 124 may generate as an output a tissue proximity output that indicates a distance between the portion of the catheter associated with the tetrapolar electrode configuration (once again, alpha or beta) and the adjacent tissue.

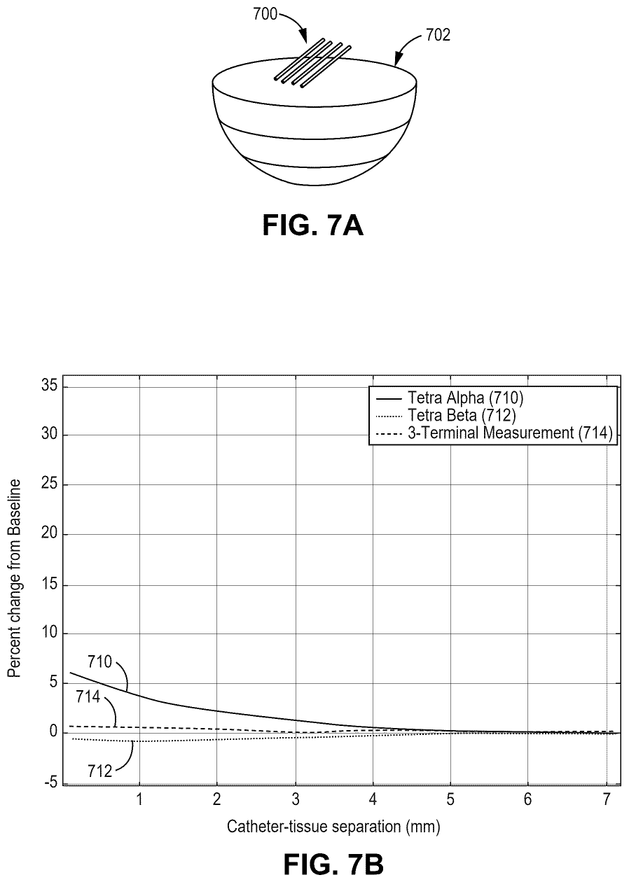

FIG. 7A is a schematic illustrating a simulated planar array of electrodes (such as those described with respect to FIGS. 4A-4F) placed at various positions relative to simulated tissue 602. In contrast with the embodiment shown in FIG. 6A in which the planar array 600 was approximately parallel with the tissue surface 602, the planar array 700 shown in FIG. 7A is positioned at an angle (e.g., 30°) to the tissue surface 702.

FIG. 7B is a graph comparing the tetrapolar alpha configuration measurements (line 710) with the tetrapolar beta configuration (line 712). In addition, a three-terminal measurement is (line 714) is provided as well. The y-axis once again illustrates the percent change from baseline of the measurement and the x-axis illustrates the distance between the planar array 700 and the tissue 702. In this embodiment, tetrapolar beta configuration 712 does not provide any percent change over the baseline as the planar array 700 is brought into contact with the tissue 702 (perhaps even a negative percent change, indicating negative sensitivity in the region containing the tissue). The three-terminal measurement illustrates almost no change as the planar array 700 is brought into contact with the tissue 702. As described above with respect to FIGS. 5A and 5B, the heterogenous sensitivity of the beta tetrapolar configuration—including the negative sensitivity profiles located on opposite sides of the tetrapolar configuration—are useful in this embodiment for determining that the array 600 is not in good alignment with the tissue surface 602. In contrast, the tetrapolar alpha configuration 710 does provide a substantial percent change over the baseline as the planar array 700 is brought into contact with the tissue 702. FIGS. 7A, 7B illustrate how the specificity/sensitivity of tetrapolar measurement depends on location and configuration of the drive electrodes and sense electrodes. In addition, these embodiments illustrate how different configurations may be utilized (either alone or in combination with one another) to glean additional information about the position/orientation of the planar array. For example, in the embodiment shown in FIGS. 7A, 7B the tissue proximity detector 124 (shown in FIG. 1) may utilize a combination of comparisons to determine the orientation of the planar array. For example, the tissue proximity detector 124 may compare the tetrapolar alpha measurement to one or more thresholds value and the tetrapolar beta measurement to one or more threshold values. In addition, the tissue proximity detector 124 may combine the results of each of these comparisons to determine one or more of tissue proximity, tissue contact and/or orientation of the planar array. In this example, if the tetrapolar alpha measurement is above a threshold and the tetrapolar beta configuration is less than a threshold, the tissue proximity detector utilizes the combination of tetrapolar measurements to determine orientation of the planar array carrying the two tetrapolar electrode configurations. That is, a tetrapolar alpha measurement at tissue contact (measuring a percent change over the baseline) and the tetrapolar beta measurement at tissue contact (measuring no percent change over the baseline) can be utilized to determine that the planar array 700 is not positioned flat or parallel with the tissue surface 702. This may be communicated to a physician/technician as a prompt to re-position the orientation of the planar array.

FIG. 8 is a graph illustrating tetrapolar measurements collected while a distal end of a medical device is positioned within a patient and displayed to a user as signal traces over time according to some embodiments. In particular, the graph illustrates the first and second tetrapolar measurements 800, 802. The first tetrapolar measurements 800 were collected from a tetrapolar alpha configuration positioned along a first spline and the second tetrapolar measurements 802 were collected from a tetrapolar alpha configuration positioned along a second spline. In this example, the first spline is located adjacent to the second spline but in other embodiments the first spline may not be adjacent to the second spline (i.e., one or more intervening splines may be located between the first and second spline). The y-axis illustrates the percent change in the tetrapolar measurement, and the x-axis illustrates time. In the first time period T₁, prior to approximately 40 seconds, the planar array is in the blood pool and both the first tetrapolar measurement 800 and the second tetrapolar measurement 802 are approximately equal. A comparison of the first and second tetrapolar measurements 800, 802 to threshold values by tissue proximity detector 124 (shown in FIG. 1) indicates that neither tetrapolar configuration of electrodes is in close proximity or contact to adjacent tissue. In a second time period T₂, extending from approximately 40 seconds to approximately 80 seconds, the planar array is brought into contact with the tissue as indicated in both the first and second tetrapolar measurements increasing by a significant amount (e.g., approximately 42%). A comparison of the first and second tetrapolar measurements 800, 802 to threshold values by tissue proximity detector 124 may indicate close proximity and/or contact between the tetrapolar configuration of electrodes and adjacent tissue. However, during the second time period the first tetrapolar measurement 800 is greater than the second tetrapolar measurement 802. This difference between the respective first and second tetrapolar measurements is utilized by the tissue proximity detector 124 to detect that the planar array is not positioned flat against the tissue. For example, in some embodiments the tissue proximity detector may compare the first tetrapolar measurement 800 to the second tetrapolar measurement 802 and utilize the detected difference in magnitude to determine that the planar array is not oriented flat against the adjacent tissue. In some embodiments, the difference is compared to one or more additional thresholds to detect and quantify the difference in magnitude between the first tetrapolar measurement 800 and the second tetrapolar measurement 802. In a third time period T₃, starting at approximately 80 seconds, the planar array is re-positioned and the second tetrapolar measurement is brought into alignment or overlap with the first tetrapolar measurement, indicating that the planar array is positioned such that both splines associated with the first and second tetrapolar configurations are in contact with the tissue. Once again, both the first and second tetrapolar measurements in time T₃ may be compared to threshold values by tissue proximity detector 124. In addition, tissue proximity detector may compare the first and second tetrapolar measurements to one another to detect the amount of overlap between the respective measurements. The amount of overlap between the first and second tetrapolar measurement required to indicate the planar array is positioned flat or substantially flat with the tissue may vary. In some embodiments, the planar array is determined to be substantially flat if the difference in the respective tetrapolar measurements is below a threshold value. In other embodiments, other methods of comparison (e.g., ratios of tetrapolar measurements to baseline tetrapolar measurements) may be utilized to determine whether the planar array including the first and second configuration of tetrapolar electrodes is oriented correctly.

This example illustrates how information from more than one tetrapolar grouping of electrodes—even if sharing the same configuration (e.g., tetrapolar alpha configuration in the example shown in FIG. 8)—can be utilized to collect additional information regarding the status of the planar array. In this embodiment, the first tetrapolar alpha configuration (line 800) would have utilized first and second drive electrodes and the second tetrapolar alpha configuration (line 802) would have utilized additional first and second drive electrodes, but as described in more detail below, in some embodiments a single pair of drive electrodes can be utilized as the basis for collecting tetrapolar measurements from a plurality of sense electrodes (i.e., more than just the first and second sense electrodes).

In addition, the example provided in FIG. 8 illustrates how a comparison of a first tetrapolar measurement to a second tetrapolar measurement (from a second set of tetrapolar electrodes) can be utilized—either alone or in combination with the tetrapolar measurements themselves—to improve or confirm the positioning of the electrode array with respect to the adjacent tissue. For example, in the embodiment shown in FIG. 8, after approximately 40 seconds both the first and second tetrapolar measurements may exceed some threshold value indicating tissue contact. In this embodiment, an additional comparison of the first and second tetrapolar measurements is utilized between 40 seconds and 80 seconds to confirm that the electrode array should be rotated to improve contact between the electrode array and tissue, which is then confirmed after 80 seconds by both the first and second tetrapolar measurements being approximately equal to one another and greater than some threshold value.

In some embodiments, the first and second tetrapolar trace measurements shown in FIG. 8 may be displayed to a user as shown. In some embodiments, information regarding tissue proximity, contact status, and/or orientation may be conveyed to the user via changes in color of the displayed tetrapolar trace measurements. For example, the color of the first and second tetrapolar trace measurements may be given a first color when no contact or close proximity to tissue is detected, and a second color when the respective tetrapolar measurements exceed a threshold value (or in other embodiments, a continuing change in colors to indicate changes in tissue proximity). In addition, the comparison of the tetrapolar measurements to one another to determine orientation of the planar array may also be expressed by changing the color of the respective traces (and/or some other visual, audio output) to provide a visual/audio indication to the user that the planar array is positioned flat against the adjacent tissue.

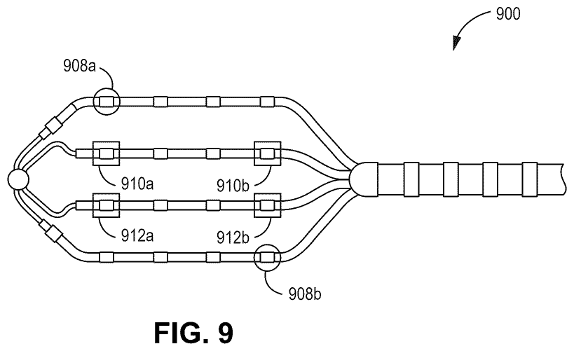

FIG. 9 is a schematic view of a distal end 900 of a medical device having an array of electrodes including a first pair of drive electrodes 908a, 908b and first and second pairs of sense electrodes 910a, 910b, and 912a, 912b according to some embodiments.

In this embodiment, the first drive electrode 908a is located at a distal end of a first spline 904a and the second drive electrode 908b is located at a proximal end of a fourth spline 904d. That is, the first and second drive electrodes 908a, 90b are located diagonally from one. The first pair of sense electrodes 910a, 910b are located on the second spline 904b. The first sense electrode 910a is located at a distal end of the second spline 904b and the second sense electrode 910b is located at a proximal end of the second spline 904b. The second pair of sense electrodes 912a, 912b are located on the third spline 904c. The third sense electrode 912a is located at a distal end of the third spline 904c and the fourth sense electrode 912b is located at a proximal end of the third spline 904c.

In some embodiments, first tetrapolar configuration provided by the location and position of the first and second drive electrodes 908a, 908b and first and second sense electrodes 910a, 910b provides first tetrapolar measurements while the second tetrapolar configuration provided by the location and position of the first and second drive electrodes 908a, 908b and third and fourth sense electrodes 912a, 912b provides second tetrapolar measurements. A benefit of utilizing a single pair of drive electrodes to generate first and second tetrapolar measurements is that additional tetrapolar measurements are provided without the expense of two additional drive electrodes. Each configuration provides different sensitivity and specificity that can be utilized to extract additional information regarding tissue proximity/contact without requiring additional drive electrodes.

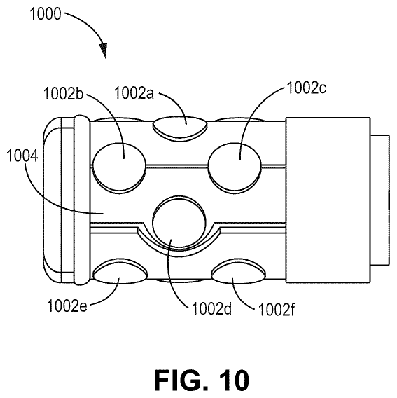

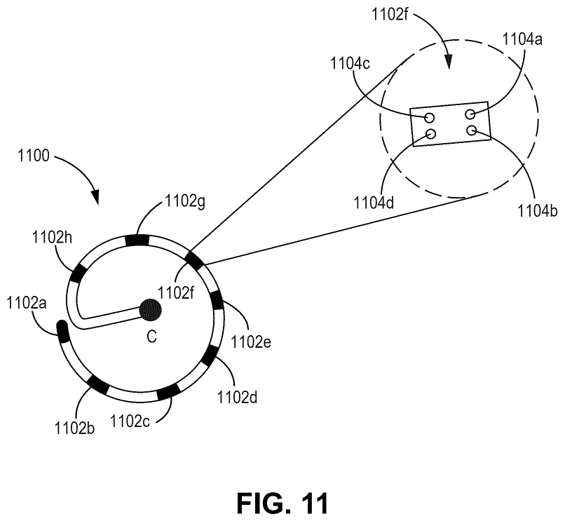

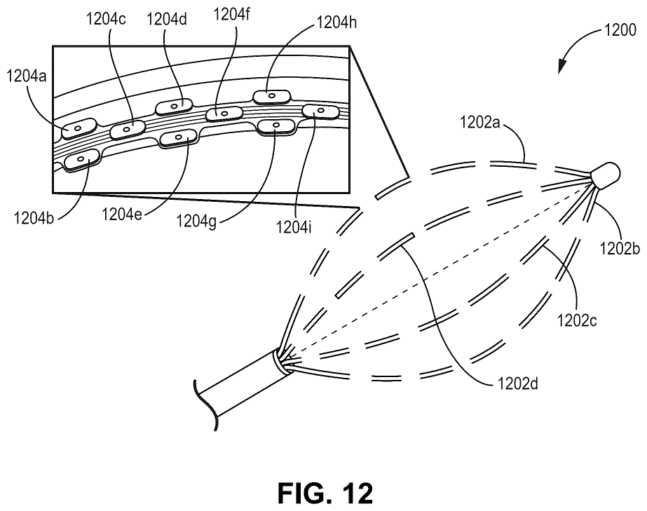

FIGS. 10-12 are schematic views of various configurations of medical device that may utilize a plurality of electrodes configured for tetrapolar measurements FIG. 10 illustrates an embodiment in which a plurality of electrodes are located circumferentially around an outer surface of the medical device and configured to collect tetrapolar measurements. FIG. 11 is a schematic view of a loop catheter having a plurality of electrode clusters spaced along the circular distal end of the catheter and configured to collect tetrapolar measurements. FIG. 12 illustrates a basket assembly comprised of a plurality of splines, with each spline including a plurality of electrodes configured to collect tetrapolar measurements.

Referring to the embodiment shown in FIG. 10, the distal end 1000 of the medical device includes a plurality of electrodes 1002a-1002f spaced around an outer surface of the distal end 1000. In some embodiments, tetrapolar measurements are taken by selecting at least two of the electrodes 1002a-1002f as drive electrodes and at least two or more of the electrodes not utilized as drive electrodes as sense electrodes. For example, in one embodiment it may be beneficial to select electrodes 1002a, 1002e as drive electrodes and utilize electrodes 1002b, 1002d as sense electrodes. In other embodiments, other configurations of drive electrodes and sense electrodes may be utilized. Similarly, the tetrapolar measurements may be utilized to detect tissue proximity/contact, sheath detection, etc.

Referring to the embodiment shown in FIG. 11, the circular or loop-shaped distal end 1100 of the medical device includes a plurality of electrode clusters 1102a-1102g (collectively, “electrode clusters 1102”) spaced along a length of the distal end 1100. In some embodiments, each electrode cluster 1102 includes four separate electrodes 1104a, 1104b, 1104c, and 1104d as illustrated in the magnified view of electrode cluster 1102f. In some embodiments, tetrapolar measurements are taken by selecting at least two of the electrodes 1104a-1104d in each electrode cluster 1102 as drive electrodes and at least two or more of the electrodes 1104a-1104d not utilized as drive electrodes as sense electrodes. For example, in one embodiment it may be beneficial to select electrodes 1104c,1104b as drive electrodes and utilize electrodes 1104a, 1104d as sense electrodes. In other embodiments, other configurations of drive electrodes and sense electrodes may be utilized. Similarly, the tetrapolar measurements measured with respect to each electrode cluster 1102a-1102g may be utilized to detect tissue proximity/contact, sheath detection, etc. with respect to that electrode cluster.

FIG. 12 is a schematic view of a basket-shaped distal end 1200 of a medical device having a plurality of splines 1202a, 1202b, 1202c, and 1202d and one or more electrodes 1204a-1204i located on each of the plurality of splines 1202a-1202d. In some embodiments, each spline may include a single electrode. In the embodiment shown in FIG. 12, each spline includes a flexible circuit 1206 that in turn includes a plurality of electrodes 1204a-1204i spaced apart from one another. In some embodiments, each spline 1202a-1202d includes a flexible circuit 1206 located on an outer surface of the spline (i.e., the surface configured to contact tissue). In other embodiments, each spline 1202a-1202d includes a flexible circuit 1206 located on an inner surface of the spline (i.e., the surface configured to remain in the blood pool), and in some embodiments each spline 1202a-1202d includes a flexible circuit 1206 located on both the outer surface and the inner surface of the spline. As described above, the plurality of electrodes 1204a-1204i are grouped into a plurality of electrode clusters, with each electrode cluster having enough electrodes to generate tetrapolar measurements. For example, in some embodiments each electrode cluster would require at least two drive electrodes and at least two sense electrodes. In some embodiments, tetrapolar measurements are taken with respect to sense electrodes located on the same spline. In other embodiments, tetrapolar measurements are taken with respect to sense electrodes located on different splines. Once again, the tetrapolar measurements may be utilized to detect tissue proximity/contact, sheath detection, etc. with respect to the plurality of electrodes and/or electrode clusters.

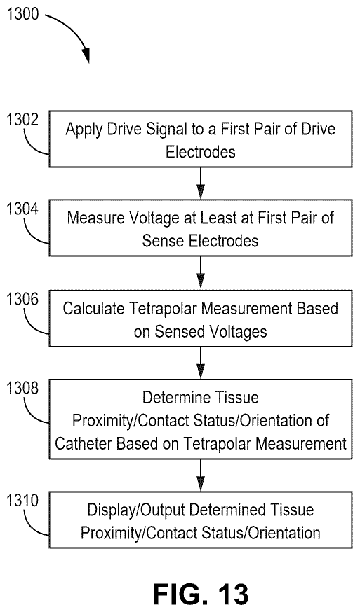

FIG. 13 is a flowchart illustrating a method 1300 of utilizing tetrapolar measurements and utilizing the tetrapolar measurements to detect tissue proximity according to some embodiments.

At step 1302, a drive signal is applied to a pair of drive electrodes included as part of a tetrapolar configuration. In some embodiments, a plurality of drive signals may be applied to a plurality of pairs of drive electrodes either sequentially or simultaneously, corresponding with a plurality of tetrapolar configurations of electrodes. In some embodiments, the drive signals are sinusoidal. In other embodiments, the drive signals may have other geometries (e.g., square wave). In some embodiments, drive signals for each pair of drive electrodes are applied at different frequencies from one another (e.g., applied at 5 kHz intervals). In some embodiments, the unique frequencies are provided at a frequency less than 30 kHz, less than 20 kHz, and less than 10 kHz. In some embodiments, the unique frequencies are provided at frequencies less than 2 kHz. The drive signal may be generated by a constant current source or a variable current source.

At step 1304, voltage is measured between at least a pair of sense electrodes, wherein the sense electrodes are separate from the drive electrodes. In some embodiments, this is measured directly utilizing a voltage differential amplifier with inputs connected to the first pair of sense electrodes. In other embodiments, the voltages are measured between the first and second sense electrodes and a reference node (e.g., associated with a reference electrode located for example on a surface patch or associated with a medical device located within the patient). In some embodiments, voltages are measured between a plurality of sense electrodes in response to a drive signal applied to the first pair of drive electrodes. In addition, at step 1304, voltages may be measured with respect to more than one tetrapolar configuration of electrodes. As discussed with respect to FIGS. 4A, 4B, the first and second tetrapolar configurations may be employed on the same device. The first and second configurations may utilize the same or different tetrapolar configuration of electrodes.

At step 1306, a tetrapolar measurement is calculated based on the two or more voltages measured at step 1304. In some embodiments, the voltage measured at step 1304 is sufficient and may be utilized as the tetrapolar measurement. For example, in the case in which a constant current source is utilized, the voltage measured between the sense electrodes may be utilized as the tetrapolar measurement without further calculation. In other embodiments, the voltage measured at step 1304 between the respective sense electrodes are divided by an amplitude of the drive current. For example, if the drive signal is provided by a variable current source, the voltage between the respective electrodes is divided by the amplitude of the current to measure an impedance value between the respective sense electrodes. In other embodiments, the tetrapolar measurement is calculated as a ratio of the voltages measured at step 1304. For example, the calculated ratio may be the voltage measured at a first electrode divided by the voltage measured at a second electrode. A plurality of tetrapolar measurements may be calculated based on the number of tetrapolar configurations of electrodes.

At step 1308 tissue proximity/contact status is determined based on the tetrapolar measurement calculated at step 1306. As discussed above, tissue proximity/contact status is not limited to detecting proximity/contact status between the sense electrodes and the adjacent tissue. Rather, the tetrapolar measurements reflect the impedance in the region surrounding the drive electrodes and the sense electrodes (based on the sensitivity of the particular configuration of electrodes employed). In this way, tissue proximity/contact status provides information regarding proximity/contact status of the catheter in general, not just of the sense electrodes. In some embodiments, tissue proximity/contact status is determined by comparing the tetrapolar measurement to one or more threshold values. In some embodiments, a tissue proximity output is provided in units of length, providing a distance between the catheter and adjacent tissue. In some embodiments, contact status is provided as a binary indication of whether the catheter is in contact with the adjacent tissue or not. In some embodiments, contact status may provide additional information beyond a binary interpretation (e.g., no contact, intermittent contact, in contact). In other embodiments, other information may be extracted from the tetrapolar measurement. For example, in some embodiments the tetrapolar measurement is utilized for sheath detection.

In some embodiments, tissue proximity/contact status is further determined based on a comparison of a first tetrapolar measurement with a second tetrapolar measurement—utilizing the same or different tetrapolar configuration. For example, the comparison of the first tetrapolar measurement with the second tetrapolar measurement—as shown in FIG. 8, for example—can be utilized to detect the catheter and associated electrodes being offset or angled with respect to the tissue when the first and second tetrapolar measurements are above a threshold, but indicate a difference in amplitude relative to one another. Likewise, a comparison of the first and second tetrapolar measurements that shows equal amplitudes (as well as the measurements being above a given threshold) can be utilized to confirm contact between the catheter and the associated electrodes and the corresponding tissue. In other embodiments, the first and second tetrapolar measurements may be generated by different tetrapolar configurations having different sensitivities, and the different sensitivities may be utilized to assess orientation of the electrode array with respect to the adjacent tissue.

At step 1310, the tissue proximity/contact status determined at step 1208 is communicated to a technician/user. For example, in some embodiments the tissue proximity/contact status is displayed graphically via a display. This may include generating a representative display of the medical device comprising the plurality of electrodes and indicating via colors/highlighting the proximity/contact status of the catheter in general as well as each of the plurality of electrodes making up the one or more tetrapolar electrode configurations. In other embodiments, the calculated tissue proximity may be utilized to make determinations regarding whether the catheter or general or an electrode in particular is positioned to deliver therapy. In this embodiment, the display may highlight or indicate those electrodes within a desired proximity for delivering therapy. In other embodiments, the information regarding tissue proximity/contact status (or sheath detection, etc.) may be communicated to a user/technician in a myriad of ways.

In some embodiments, the tetrapolar measurements themselves are displayed to a technician/user. In some embodiments, the tetrapolar measurements are displayed to the technician/user as a waveform over time (e.g., as shown in FIG. 8). In some embodiments, the technician/user analyzes the displayed waveforms to detect tissue proximity, contact status, and/or orientation of the catheter. In other embodiments, the display of the waveforms may be color-coded to indicate tissue proximity/contact status. For example, the color of the waveforms may change to indicate tissue proximity/contact status. In other embodiments, various other types of displays may be utilized to indicate tissue proximity/status.

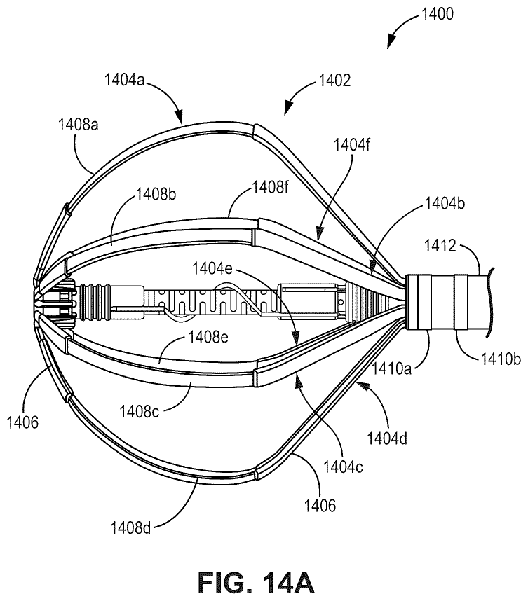

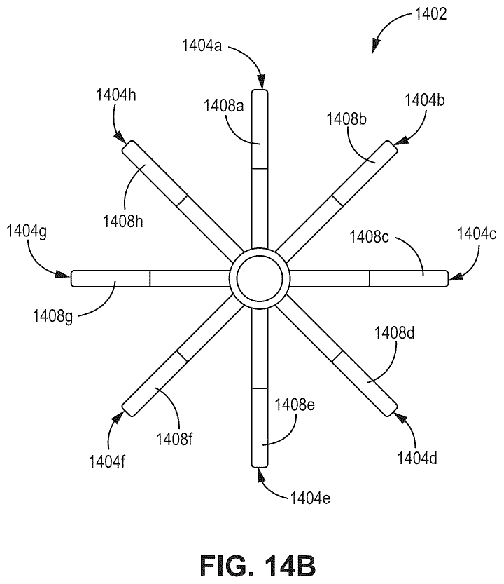

FIGS. 14A and 14B are schematic views of a distal end 1402 of a medical device 1400 having a basket geometry that includes a plurality of splines 1404a, 1404b, 1404c, 1404d, 1404e, 1404f, 1404g, and 1404h and a single electrode 1408 on each spline configured for tetrapolar measurements according to some embodiments. FIG. 14A is a side view of the distal end 1402 (in which only splines 1404a-1404f are visible) and FIG. 14B is an end view of the distal end 1402 (in which each spline 1404a-1404h is visible). In one example, each spline 1404a-1404h comprises a conductive material (e.g., nitinol) and insulative material 1406 located over at least portions of the spline 1404a-1404h (e.g., in this example, the insulative material is placed over proximal and distal portions of each spline 1404a-1404h). The electrodes 1408a-1408h is the portion of the spline 1404a-1404h that does not include insulation 1406.

In some examples, the electrodes 1408a-1408h located on each of the plurality of splines 1404a-1404h, respectively, are configured to deliver pulsed-field ablation (PFA) therapy. In some examples, the plurality of electrodes 1408a-1408h may also be utilized to measure tetrapolar impedances. As discussed above, tetrapolar impedances require two drive electrodes and two sense electrodes. In one example, electrodes 1408a and 1408d are utilized as drive electrodes and electrodes 1408b and 1408c are utilized as sense electrodes (i.e., sense electrodes are located inside to the drive electrodes). In this example, electrodes 1408h and 1408e may also be utilized as drive electrodes and electrodes 1408g and 1408f are utilized as sense electrodes (i.e., sense electrodes are located inside to the drive electrodes). In other examples, electrodes 1408a and 1408e are utilized as drive electrodes and some combination of electrodes 1408b, 1408c, 1408d, 1408f, 1408g, and 1408h are utilized as sense electrodes. This configuration is similar to that shown in FIG. 4A. In another example, the electrodes 1408c and 1408d are utilized as the drive electrodes and electrodes 1408a and 1408b are utilized as the sense electrodes, similar to the embodiment shown in FIG. 4B. Other combinations of electrodes may be utilized, for example electrodes 1408f and 1408g could be utilized as drive electrodes and electrodes 1408h and 1408e could be utilized as sense electrodes. In still other examples, the electrodes 1408a and 1408c are utilized as the drive electrodes and electrodes 1408b and 1408d are utilized as the sense electrodes. In other examples, by virtue of the electrical reciprocity principle, the drive and sense electrodes just described can be swapped, yielding the same impedance and advantages. In still other examples, configurations may be utilized in which electrodes are a part of more than one tetrapolar measurement.

In another example, the distal end 1402 of the medical device 1400 includes ring electrodes 1410a and 1410b located proximal of the splines 1404a-1404f making up the basket assembly. In some examples, the rings electrodes 1410a, 1410b may be utilized as drive electrodes and two or more of the spline electrodes 1408a-1408f are utilized as sense electrodes.

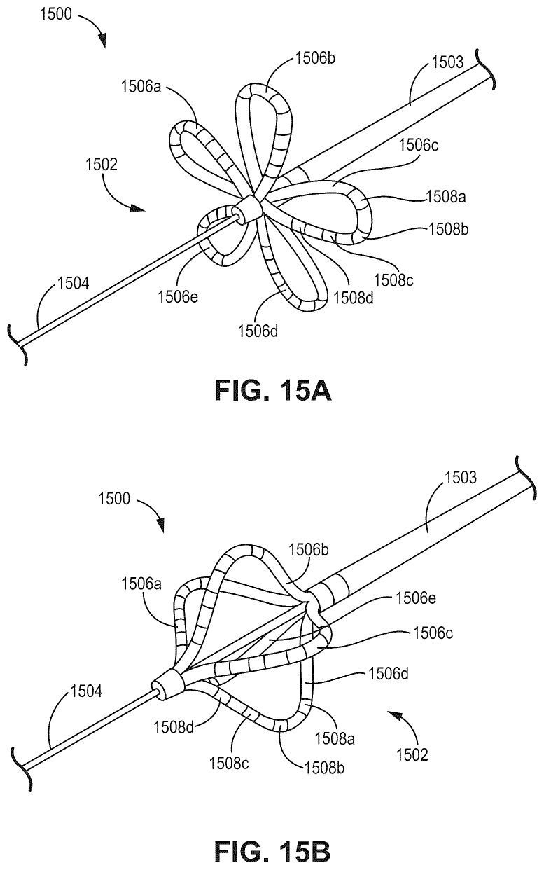

FIGS. 15A and 15B are schematic views of a distal end 1502 of a medical device 1500 having a plurality of splines 1506a-1506e, each spline including four electrodes. For the sake of simplicity, only electrodes 1508a, 1508b, 1508c, and 1508dprovided on spline 1506c are labeled. In FIG. 15A, the splines are deformed into a ‘flower’ configuration and in FIG. 15B, the splines are deformed into a ‘basket’ configuration. In one example, electrodes 1508a and 1508d are utilized as drive electrodes and electrodes 1508b and 1508c are utilized as sense electrodes. This is similar to the configuration shown in FIG. 4A in which the sense electrodes 410a, 410b are located within the drive electrodes 408a, 408b. In other examples, other configuration of drive and sense electrodes may be utilized. For example, electrodes 1508a and 1508b may be utilized as drive electrodes and electrodes 1508c and 1508d may be utilized as sense electrodes. In still other examples, electrodes 1508a and 1508c are utilized as drive electrodes and electrodes 1508b and 1508d are utilized as sense electrodes.

In some embodiments, drive and sense electrodes may be provided on two or more splines. For example, electrodes located on a first spline (e.g., 1506a) may be utilized as drive electrodes and electrodes location on a second spline (e.g., 1506b) may be utilized as sense electrodes. In other examples, other combinations of electrodes on various splines may be utilized depending on the type of information to be extracted.

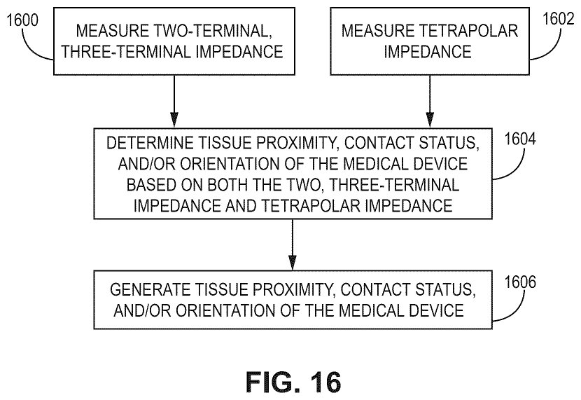

FIG. 16 is a flowchart illustrating a method of utilizing a combination of both tetrapolar measurements in combination with typical tissue impedance measurements (two-terminal or three-terminal measurements) to detect tissue proximity according to some embodiments. At step 1600, two-terminal or three-terminal impedances are measured. Two-terminal impedance measurements apply a drive signal to a first electrode located on a distal end of a medical device and measures a voltage between the first electrode and a reference electrode (e.g., reference electrode may be a patch electrode located on the skin of the patient). A return path to the drive circuitry is provided through the reference electrode and the sense circuitry. FIG. 2 described above illustrates a three-terminal measurement circuit, in which a drive signal is applied between first and second electrodes E₁ and E₂ and voltages are measured between each electrode and a reference electrode 214 (hence the name, three-terminal device) by comparators 212a, 212b, respectively.

At step 1602, tetrapolar impedances are measured. As described above, a tetrapolar impedance measurement requires applying a drive signal to a pair of drive electrodes, and a measuring a voltage between a pair of sense electrodes that are separate from the drive electrodes. The tetrapolar impedance is calculated based on the voltage sensed between the pair of sense electrodes. In contrast with the two-terminal and three-terminal impedance measurement, the tetrapolar impedance measurement requires at least four electrodes. In some examples, the first and second sense electrodes utilized in the tetrapolar measurement correspond with the first and second electrodes utilized in the three-terminal measurement or the at least one electrode utilized in the two-terminal measurement. In this example, the drive signal utilized for the two or three-terminal impedance measurement is separate from the drive signal utilized for the tetrapolar measurement. The drive signals may be applied at different frequencies or time-multiplexed to avoid interference with one another.

In other examples, the first and second drive electrodes utilized in the tetrapolar measurement correspond with the first and second electrodes utilized in the three-terminal measurement or the at least one electrodes utilized in the two-terminal measurement. In this example, the drive signal utilized in the tetrapolar measurement is the same drive signal utilized in the two-terminal or three-terminal measurement.

At step 1604, tissue proximity/contact status is determined based on both the two-terminal, three-terminal measurement calculated at step 1600 and the tetrapolar measurement calculated at step 1602. In some examples, the two-terminal, three-terminal measurements provides high resolution/high sensitivity tissue proximity/contact when the electrode is relatively close to the tissue (e.g., less than 1 mm), whereas the tetrapolar measurements provides high resolution/high sensitivity (as compared to the two-terminal, three-terminal measurement) when further from the tissue (e.g., greater than 1 mm). In some examples, step 1604 utilizes both measurements to improve the assessment of tissue proximity/contact. For example, if the two-terminal, three-terminal measurement indicates that the electrode is not in contact with the tissue and provides a tissue proximity estimate of greater than 1 mm, then more weight will be given to the tissue proximity estimate provided by the tetrapolar measurement, which provides better resolution/sensitivity when the electrode is not in contact with the tissue. Conversely, if the two-terminal, three-terminal measurement indicates that the electrode is in contact with the tissue (i.e., tissue proximity of less than 1 mm), then more weight is given to the two-terminal, three-terminal measurement than to the tetrapolar measurement.

In some embodiments, rather than measure both a two-terminal, three-terminal measurement and a tetrapolar measurement, the method may include measuring ONLY the tetrapolar measurement so long the tetrapolar measurement indicates that the electrode(s) are not in contact with the tissue or are at least some distance from the tissue. When the tetrapolar measurement indicates that the electrode(s) are within some threshold distance of the tissue (e.g., 2 mm, 1 mm, etc.) then two-terminal, three-terminal measurements are taken to provide the desired higher resolution/higher sensitivity provided by two-terminal, three-terminal measurements when in close proximity with tissue (e.g., less than 1 mm). In this example, so long as the electrode is in contact or at least very close proximity to the tissue, only two-terminal, three-terminal measurements would be taken until those measurements indicate that the tissue proximity has exceeded some threshold amount (e.g., greater than 2 mm).

At step 1606, the tissue proximity/contact status determined at step 1604 is communicated to a technician/user. For example, in some embodiments the tissue proximity/contact status is displayed graphically via a display. This may include generating a representative display of the medical device comprising the plurality of electrodes and indicating via colors/highlighting the proximity/contact status of the catheter in general as well as each of the plurality of electrodes making up the one or more tetrapolar electrode configurations. In other embodiments, the calculated tissue proximity may be utilized to make determinations regarding whether the catheter or general or an electrode in particular is positioned to deliver therapy. In this embodiment, the display may highlight or indicate those electrodes within a desired proximity for delivering therapy. In other embodiments, the information regarding tissue proximity/contact status (or sheath detection, etc.) may be communicated to a user/technician in a myriad of ways.

In some embodiments, the tetrapolar measurements and/or two-terminal, three-terminal measurements themselves are displayed to a technician/user. In some embodiments, the tetrapolar measurements and/or two-terminal, three-terminal measurements are displayed to the technician/user as a waveform over time (e.g., as shown in FIG. 8). In some embodiments, the technician/user analyzes the displayed waveforms to detect tissue proximity, contact status, and/or orientation of the catheter. In other embodiments, the display of the waveforms may be color-coded to indicate tissue proximity/contact status. For example, the color of the waveforms may change to indicate tissue proximity/contact status. In other embodiments, various other types of displays may be utilized to indicate tissue proximity/status.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

Clause 1. A medical device system comprising: a medical device comprising: a proximal end; a distal end; a pair of drive electrodes located at the distal end; and a first pair of sense electrodes located at the distal end, the first pair of sense electrodes being separate from the pair of drive electrodes; drive circuitry configured to provide a drive signal to the pair of drive electrodes; and sense circuitry connected to the first pair of sense electrodes to sense a voltage generated in response to the drive signal provided to the pair of drive electrodes and to calculate a first tetrapolar measurement in response to the sensed voltage, the first tetrapolar measurement being indicative of tissue proximity, contact status, and/or orientation of the distal end of the medical device.

Clause 2. The medical device system of clause 1, wherein the first tetrapolar measurement is displayed to a user.

Clause 3. The medical device system of clause 2, wherein the first tetrapolar measurement is displayed to the user as a signal trace that is color-coded to provide a visual indication of contact status.

Clause 4. The medical device system of clause 1, further including: a tissue proximity detector configured to generate an output representative of tissue proximity, contact status, and/or orientation of the distal end based on the first tetrapolar measurement.

Clause 5. The medical device system of clause 4, wherein the tissue proximity detector generates outputs representative of tissue proximity, contact status, and/or orientation of the distal end based on comparisons of the first tetrapolar measurement to one or more thresholds.

Clause 6. The medical device system of clause 4, further including a second pair of sense electrodes utilized to generate a second tetrapolar measurement, wherein the tissue proximity detector generates an output representative of tissue proximity, contact status, and/or orientation of the distal end based on a comparison of the first tetrapolar measurement to the second tetrapolar measurement.

Clause 7. The medical device system of clause 1, wherein the drive circuitry is a constant current source and the first tetrapolar measurement is based on the sensed voltage.

Clause 8. The medical device system of clause 1, wherein the drive circuitry is a variable current source that delivers a variable current to the pair of drive electrodes, and wherein the first tetrapolar measurement is a tetrapolar impedance measurement calculated based on the sensed voltage and a magnitude of the variable current.

Clause 9. The medical device system of clause 1, wherein the first pair of sense electrodes includes a first sense electrode and a second sense electrode, wherein the second sense electrode is located adjacent to the first sense electrode.

Clause 10. The medical device system of clause 9, wherein the first pair of drive electrodes includes a first drive electrode and a second drive electrode, the first drive electrode located on a first side of the first pair of sense electrodes and the second drive electrode located on a second side of the first pair of sense electrodes.

Clause 11. The medical device system of clause 1, wherein the first pair of drive electrodes includes a first drive electrode and a second drive electrode, wherein the second drive electrode is located adjacent to the first drive electrode.

Clause 12. The medical device system of clause 11, wherein the second drive electrode is located adjacent to the first sense electrode.

Clause 13. The medical device system of clause 1, wherein the medical device further includes one or more additional pairs of sense electrodes and wherein the sense circuitry is additionally connected to the one or more additional pairs of sense electrodes to measure additional tetrapolar measurements in response to the drive signal provided to the first pair of drive electrodes.

Clause 14. The medical device system of clause 1, wherein the medical device includes a circular distal end, wherein the pair of drive electrodes and the pair of sense electrodes are located on the circular distal end.

Clause 15. The medical device system of clause 1, wherein the medical device includes a basket distal end comprised of a plurality of splines, wherein the pair of drive electrodes and the pair of sense electrodes are located on one or more of the plurality of splines.

Clause 16. The medical device system of clause 1, wherein the drive electrodes and the sense electrodes are arranged in a configuration having known distances between the drive electrodes and the sense electrodes.

Clause 17. A medical device system comprising: a medical device comprising: a first spline having at least a first pair of drive electrodes and a first pair of sense electrodes; and a second spline having at least a second pair of drive electrodes and a second pair of sense electrodes; and drive circuitry configured to provide a first drive signal to the first pair of drive electrodes and a second drive signal to the second pair of drive electrodes; and sense circuitry connected to the first pair of sense electrodes to measure a first tetrapolar measurement generated in response to the first drive signal provided to the first pair of drive electrodes, the sense circuitry connected to the second pair of sense electrodes to measure a second tetrapolar measurement generated in response to the second drive signal provided to the second pair of drive electrodes.

Clause 18. The medical device system of clause 17. wherein the first and second tetrapolar measurements are displayed to a user.

Clause 19. The medical device system of clause 18, wherein the first and second tetrapolar measurements are displayed to the user as first and second signal traces, respectively, wherein the first and second signal traces are color-coded to provide a visual indication of proximity, contact status, and/or orientation of the medical device.

Clause 20. The medical device system of clause 17, further including: a tissue proximity detector configured to determine tissue proximity based on the first and second tetrapolar measurements.

Clause 21. The medical device of clause 20, wherein the tissue proximity detector determines tissue proximity, contact status, and/or orientation of the medical device based, at least in part, on comparisons of the first tetrapolar measurement and the second tetrapolar measurement to one or more threshold values.

Clause 22. The medical device of clause 21, wherein the tissue proximity detector determines tissue proximity, contact status, and/or orientation of the medical device based, in addition, on comparisons of the first tetrapolar measurement to the second tetrapolar measurement.

Clause 23. The medical device of clause 17, wherein the first pair of drive electrodes and the first pair of sense electrodes are arranged in a first configuration and wherein the second pair of drive electrodes and the second pair of sense electrodes are arranged in a second configuration different from the first configuration.

Clause 24. The medical device of clause 17, wherein the first pair of drive electrodes are located adjacent to one another and the first pair of sense electrodes are located adjacent to one another.

Clause 25. The medical device of clause 17, wherein the first pair of sense electrodes are located adjacent to one another and the first pair of drive electrodes are separated from one another and located on opposite sides of the first pair of sense electrodes.

Clause 26. A method of detecting tissue proximity of a medical device, the method comprising: applying a drive signal to a first pair of drive electrodes located on the medical device; measuring a voltage between a first pair of sense electrodes located on the medical device, wherein the first pair of sense electrodes are separate from the first pair of drive electrodes, wherein the first pair of sense electrodes and the first pair of drive electrodes represent a tetrapolar configuration; calculating a first tetrapolar measurement based on the voltage measured between the first pair of sense electrodes; determining tissue proximity of the medical device based on the first tetrapolar measurement; and displaying the determined tissue proximity.

Clause 27. The method of clause 26, wherein the tetrapolar measurement represents an impedance associated with a region adjacent to the tetrapolar configuration of drive electrodes and sense electrodes located on the medical device.

Clause 28. The method of clause 26, further including: measuring a plurality of voltages associated with a plurality of pairs of sense electrodes; and calculating a plurality of tetrapolar measurements based on the plurality of measured voltages.

Clause 29. The method of clause 28, wherein determining tissue proximity of the medical device further includes determining an orientation of the medical device based on the plurality of tetrapolar measurements.

Clause 30. The method of clause 26, wherein determining tissue proximity of the first pair of sense electrodes includes calculating a distance from the tetrapolar configuration of drive electrodes and sense electrodes to the tissue based on a magnitude of the tetrapolar measurement.

Clause 31. The method of clause 26, wherein displaying the determined tissue proximity to the user includes displaying the first tetrapolar measurement to a user as a first signal trace, wherein the first signal trace is color-coded to provide a visual indication of proximity to adjacent tissue.

1. A medical device system comprising:
a medical device comprising:
a proximal end;
a distal end;
a pair of drive electrodes located at the distal end; and
a first pair of sense electrodes located at the distal end, the first pair of sense electrodes being separate from the pair of drive electrodes;
drive circuitry configured to provide a drive signal to the pair of drive electrodes; and
sense circuitry connected to the first pair of sense electrodes to sense a voltage generated in response to the drive signal provided to the pair of drive electrodes and to calculate a first tetrapolar measurement in response to the sensed voltage, the first tetrapolar measurement being indicative of tissue proximity, contact status, and/or orientation of the distal end of the medical device.

2. The medical device system of claim 1, wherein the first tetrapolar measurement is displayed to the user as a signal trace that is color-coded to provide a visual indication of contact status.

3. The medical device system of claim 1, further including:
a tissue proximity detector configured to generate an output representative of tissue proximity, contact status, and/or orientation of the distal end based on comparisons of the first tetrapolar measurement to one or more thresholds.

4. The medical device system of claim 1, further including:
a second pair of sense electrodes utilized to generate a second tetrapolar measurement; and
a tissue proximity detector configured to generate an output representative of tissue proximity, contact status, and/or orientation of the distal end based on a comparison of the first tetrapolar measurement to the second tetrapolar measurement.

5. The medical device system of claim 1, wherein the first pair of sense electrodes includes a first sense electrode and a second sense electrode, wherein the second sense electrode is located adjacent to the first sense electrode.

6. The medical device system of claim 5, wherein the first pair of drive electrodes includes a first drive electrode and a second drive electrode, the first drive electrode located on a first side of the first pair of sense electrodes and the second drive electrode located on a second side of the first pair of sense electrodes.

7. The medical device system of claim 1, wherein the first pair of drive electrodes includes a first drive electrode and a second drive electrode, wherein the second drive electrode is located adjacent to the first drive electrode.

8. The medical device system of claim 7, wherein the second drive electrode is located adjacent to the first sense electrode.

9. The medical device system of claim 1, wherein the medical device further includes one or more additional pairs of sense electrodes and wherein the sense circuitry is additionally connected to the one or more additional pairs of sense electrodes to measure additional tetrapolar measurements in response to the drive signal provided to the first pair of drive electrodes.

10. A medical device system comprising:
a medical device comprising:
a first spline having at least a first pair of drive electrodes and a first pair of sense electrodes; and
a second spline having at least a second pair of drive electrodes and a second pair of sense electrodes; and
drive circuitry configured to provide a first drive signal to the first pair of drive electrodes and a second drive signal to the second pair of drive electrodes; and
sense circuitry connected to the first pair of sense electrodes to measure a first tetrapolar measurement generated in response to the first drive signal provided to the first pair of drive electrodes, the sense circuitry connected to the second pair of sense electrodes to measure a second tetrapolar measurement generated in response to the second drive signal provided to the second pair of drive electrodes.

11. The medical device system of claim 10, wherein the first and second tetrapolar measurements are displayed to the user as first and second signal traces, respectively, wherein the first and second signal traces are color-coded to provide a visual indication of proximity, contact status, and/or orientation of the distal end of the medical device.

12. The medical device system of claim 10, further including:
a tissue proximity detector configured to determine tissue proximity, contact status, and/or orientation of the distal end of the medical device based, at least in part, on comparisons of the first tetrapolar measurement and the second tetrapolar measurement to one or more threshold values.

13. The medical device system of claim 10, further including:
a tissue proximity detector configured to determine tissue proximity, contact status, and/or orientation of the distal end of the medical device based, at least in part, on comparisons of the first tetrapolar measurement to the second tetrapolar measurement.

14. The medical device system of claim 10, wherein the first pair of drive electrodes and the first pair of sense electrodes are arranged in a first configuration and wherein the second pair of drive electrodes and the second pair of sense electrodes are arranged in a second configuration different from the first configuration.

15. The medical device system of claim 10, wherein the first pair of drive electrodes are located adjacent to one another and the first pair of sense electrodes are located adjacent to one another.

16. The medical device system of claim 10, wherein the first pair of sense electrodes are located adjacent to one another and the first pair of drive electrodes are separated from one another and located on opposite sides of the first pair of sense electrodes.

17. A method of detecting tissue proximity of a medical device, the method comprising:
applying a drive signal to a first pair of drive electrodes located on the medical device;
measuring a voltage between a first pair of sense electrodes located on the medical device, wherein the first pair of sense electrodes are separate from the first pair of drive electrodes, wherein the first pair of sense electrodes and the first pair of drive electrodes represent a tetrapolar configuration;
calculating a first tetrapolar measurement based on the voltage measured between the first pair of sense electrodes;
determining tissue proximity of the medical device based on the first tetrapolar measurement; and
displaying the determined tissue proximity.

18. The method of claim 17, wherein the tetrapolar measurement represents an impedance associated with a region adjacent to the tetrapolar configuration of drive electrodes and sense electrodes located on the medical device.

19. The method of claim 17, further including:
measuring a plurality of voltages associated with a plurality of pairs of sense electrodes; and
calculating a plurality of tetrapolar measurements based on the plurality of measured voltages.

20. The method of claim 19, wherein determining tissue proximity of the medical device further includes determining an orientation of the medical device based on the plurality of tetrapolar measurements and calculating a distance from the tetrapolar configuration of drive electrodes and sense electrodes to the tissue based on a magnitude of the tetrapolar measurement.
